SEQUENCE LISTING (14 sequences)

SEQ ID NO: 1 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 238 aa, synthetic construct
EVDKKVEPKSCDKTHTCPPCPAPVPTGGPLVFLFPPKPKDTLMISRTPEVTCVVVDVDPEDPEVKFNWYVDGVEVHNAKTKPREEQYNGAYRVVSVLTVLHQDWLNGKEYKCKVSNKLAPAPQEVTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 232 aa, synthetic construct
EPKSCDKTHTCPPCPAPVDFRDPLVFLFPPKPKDTLMISRTPEVTCVVVDVDPEDPEVKFNWYVDGVEVHNAKTKPREEQYNGAYRVVSVLTVLHQDWLNGKEYKCKVSNKLAPHPIIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 232 aa, synthetic construct
EPKSCDKTHTCPPCPAPVDFRDPLVFLFPPKPKDTLMISRTPEVTCVVVDVDPEDPEVKFNWYVDGVEVHNAKTKPQEEQYNGAYRVVSVLTVLHQDWLNGKEYKCKVSNKLAPHPIIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 232 aa, synthetic construct
EPKSCDKTHTCPPCPAPVDFRDPLVFLFPPKPKDTLMISRTPEVTCVVVDVSPEDPEVKFNWYVDGVEVHNAKTKPQEEQYNGAYRVVSVLTVLHQDWLNGKEYKCKVSNKLAPHPIIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 18 aa, synthetic construct
PTLYNVSLVMSDTAGTCY

SEQ ID NO: 7 - protein, 250 aa, synthetic construct
EPKSCDKTHTCPPCPAPVDFRDPLVFLFPPKPKDTLMISRTPEVTCVVVDVSPEDPEVKFNWYVDGVEVHNAKTKPQEEQYNGAYRVVSVLTVCHQDWLNGKEYKCKVSNKLAPHPIIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 8 - protein, 250 aa, synthetic construct
EPKSCDKTHTCPPCPAPVDFRDPLVFLFPPKPKDTLMISRTPEVTCVVVDVSPEDPEVKFNWYVDGVEVHNAKTKPQEEQYNSTYRVVSVLTVCHQDWLNGKEYKCKVSNKLAPHPIIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 9 - protein, 18 aa, synthetic construct
PTLYNVSLIMSDTGGTCY

SEQ ID NO: 10 - protein, 18 aa, synthetic construct
PTLYNVSLIMSDTAGTCY

SEQ ID NO: 11 - protein, 18 aa, synthetic construct
PTLYNVSLVMSDTGGTCY

SEQ ID NO: 12 - protein, 250 aa, synthetic construct
EPKSCDKTHTCPPCPAPVDFRDPLVFLFPPKPKDTLMISRTPEVTCVVVDVDPEDPEVKFNWYVDGVEVHNAKTKPREEQYNGAYRVVSVLTVCHQDWLNGKEYKCKVSNKLAPHPIIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGNCY

SEQ ID NO: 13 - protein, 250 aa, synthetic construct
EPKSCDKTHTCPPCPAPVDFRDPLVFLFPPKPKDTLMISRTPEVTCVVVDVDPEDPEVKFNWYVDGVEVHNAKTKPREEQYNSTYRVVSVLTVCHQDWLNGKEYKCKVSNKLAPHPIIKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGNCY

SEQ ID NO: 14 - protein, 250 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

(19) United States
(12) Patent Application Publication
(10) Pub. No.: US 2024/0010742 A1
(43) Pub. Date: Jan. 11, 2024

(54) ENGINEERED FCRIIB SELECTIVE IGG1 FC VARIANTS AND USES THEREOF

(71) Applicant: Research Development Foundation, Carson City, NV (US)

(72) Inventors: George Georgiou, Austin, TX (US); Jin Eyun Kim, Austin, TX (US); Chang-Han Lee, Austin, TX (US); George Delidakis, Austin, TX (US)

(73) Assignee: Research Development Foundation, Carson City, NV (US)

(21) Appl. No.: 18/332,571

(22) Filed: Jun. 9, 2023

Related U.S. Application Data
(60) Provisional application No. 63/351,282, filed on Jun. 10, 2022.
(60) Provisional application No. 63/385,877, filed on Dec. 2, 2022.

(51) Int. Cl. C07K 16/28

(52) U.S. Cl. CPC C07K 16/2878; C07K 2317/52; C07K 2317/41; C07K 2317/75; C07K 2317/732; C07K 2317/92; C07K 2317/71; C07K 2317/24

(57) ABSTRACT

In some aspects, mutant or variant Fc domains are provided that can exhibit increased affinity or selectivity for FcγRIIB. The variant Fc domain may be a mutant IgG1 Fc domain. In some embodiments, a mutant or variant Fc domain may be present in a therapeutic antibody such as, e.g., an agonistic antibody. Additional methods for using and identifying mutant Fc domains are also provided.

### PRIORITY

This application claims the benefit of United States Provisional Patent Application Nos. 63/351,282 filed Jun. 10, 2022, and 63/385,877 filed Dec. 2, 2022 both of which are incorporated herein by reference in their entireties.

### SEQUENCE LISTING INCORPORATION

This application contains a Sequence Listing XML, which has been submitted electronically and is hereby incorporated by reference in its entirety. Said XML Sequence Listing, created on Jun. 6, 2023, is named CLFRP0495WO.xml and is 15,515 bytes in size.

### BACKGROUND OF THE INVENTION

### 1. Field of the Invention

The present invention relates generally to the field of protein engineering. More particularly, it concerns improved compositions of Fc antibody domains conferring high binding to FcγRIIB and altered effector function.

### 2. Description of Related Art

A variety of antibodies have been developed for therapeutic purposes. The top 25 marketed recombinant therapeutic antibodies currently have well over $43.5 billion/year of sales. With a forecasted annual growth rate of 9.2% from 2010 to 2015, they are projected to increase to $62.7 billion/year by 2015 (Elvin et al., 2013). Monoclonal antibodies (mAbs) comprise the majority of recombinant proteins currently in the clinic, with 1064 products undergoing company-sponsored clinical trials in the USA or EU, of which 164 are phase III (Elvin et al., 2013). The mAb market is heavily focused on oncology and inflammatory disorders, and products within these therapeutic areas are set to continue to be the key growth drivers over the forecast period. As a group, genetically engineered mAbs generally have a higher probability of FDA approval success than small-molecule drugs. At least 50 biotechnology companies and all major pharmaceutical companies have active antibody discovery programs in place. The original method for isolation and production of mAbs was first reported in 1975 by Milstein and Kohler (Kohler and Milstein, 1975). It involved the fusion of mouse lymphocyte and myeloma cells, yielding mouse hybridomas. Therapeutic murine mAbs entered the clinical study in the early 1980s; however, problems with lack of efficacy and rapid clearance due to patients' production of human anti-mouse antibodies (HAMA) became apparent. These are and the time and cost consumption related to the technology became driving forces for the evolution of mAb production technology. Polymerase Chain Reaction (PCR) facilitated the cloning of monoclonal antibody genes directly from lymphocytes of immunized animals and the expression of combinatorial libraries of antibody fragments in bacteria (Orlandi et al., 1989). Later libraries were created entirely by in vitro cloning techniques using naive genes with rearranged complementarity determining region 3 (CDR3) (Griffiths and Duncan, 1998; Hoogenboom et al., 1998). As a result, the isolation of antibody fragments with the desired specificity was no longer dependent on the immunogenicity of the corresponding antigen. These advantages have facilitated the development of antibody fragments to several unique antigens, including small molecular compounds (haptens) (Hoogenboom and Winter, 1992), molecular complexes (Chames et al., 2000), unstable compounds (Kjaer et al., 1998), and cell surface proteins (Desai et al., 1998).

One method for screening large combinatorial libraries of antibodies to identify clones that bind to a ligand with desired affinity involves expression and display of antibody fragments or full length antibodies on the surface of bacterial cells and more specifically E. coli. Cells displaying antibodies or antibody fragments are incubated with a solution of fluorescently labeled ligand and those cells that bind the ligand by virtue of the displayed antibody on their surface are isolated by flow cytometry. In particular, Anchored Periplasmic Expression (APEx) is based on anchoring the antibody fragment on the periplasmic face of the inner membrane of E. coli followed by disruption of the outer membrane, incubation with fluorescently-labeled target, and sorting of the spheroplasts (U.S. Pat. No. 7,094,571, Harvey et al., 2004; Harvey et al., 2006).

The receptors for the Fc domain of antibodies are expressed on diverse immune cells and are important in both promoting and regulating the immunological response to antibody antigen complexes (called immune complexes). The binding of the Fc region of antibodies that have formed immune complexes with a pathogenic target cell to different Fc receptors expressed on the surface of leukocytes to elicit antibody-dependent cell cytotoxicity (ADCC) and antibody-dependent cell phagocytosis (ADCP) or complement-mediated reactions including complement dependent cytotoxicity (CDC).

In humans there are two general classes of FcγRs for IgG class antibodies: activating receptors, characterized by the presence of a cytoplasmic immunoreceptor tyrosine-based activation motif (ITAM) sequence associated with the receptor, and the inhibitory receptor, characterized by the presence of an immunoreceptor tyrosine-based inhibitory motif (ITIM) sequence (Daeron, 1997 and Bolland et al., 1999). Of note, activating FcγRs, FcγRI, FcγRIIA, FcγRIIIA, and FcγRIIIB induce activating or pro-inflammatory responses, while inhibitory FcγRIIB induces anti-inflammatory or inhibitory responses. Among activating FcγRs, FcγRIIA and FcγRIIIA have natural allotypes which can affect the binding capacity of IgG. FcγRIIA_{H131} showed higher binding affinity than FcγRIIA_{R131} for IgG and FcγRIIIA_{V158} showed higher binding affinity than FcγRIIIA_{F158} for IgG. All naturally produced antibodies and recombinant glycosylated antibodies produced by tissue culture contain Fc domains that bind to both activating and inhibitory FcγRs. (Boruchov et al. 2005; Kalergis et al., 2002).

As mentioned above, aglycosylated antibodies do not display any detectable binding to FcγRIIB. Due to the physiological importance of Fc binding to FcγRIIB and the importance of Fc binding to FcγRIIB with therapeutic antibodies (e.g., agonistic antibodies), there is a clear need for new Fc domains, such as aglycosylated Fc domains that can selectively bind FcγRIIB.

### SUMMARY OF THE INVENTION

The present disclosure overcomes limitations in the prior art by providing Fc domain variants which selectively bind FcγRIIB, without inducing activating FcγR or triggering pro-inflammatory effector functions such as antibody mediated phagocytosis or cell cytotoxicity (ADCP and ADCC respectively). In contrast to previously generated Fc mutants, in some embodiments the engineered Fc provided herein confer exquisitely selective binding to FcγRIIB and no binding or drastically impaired binding to activating receptors (FcγRI, FcγRIIa_{H131}, FcγRIIa_{R131}, FcγRIIIa_{F158}, FcγRIIIa_{V158}) and C1q. Additionally, the FcγRIIB specific mutants disclosed herein bind to the neonatal receptor FcRn in a pH dependent manner comparable to wild-type Fc domains. The mutant Fc domains may be glycosylated or aglycosylated. The ability for the mutant Fc to selectively bind FcγRIIB without inducing activating FcγR or causing antibody mediated phagocytosis may be particularly advantageous for a variety of therapies, for example including therapeutic applications where inflammatory effects mediated by antibodies due to the binding of activating FcγRs or complement need to be minimized or suppressed. Disclosed mutant Fc to selectively bind FcγRIIB include SEQ ID NOs:2-5, preferably SEQ ID NO:5. Hexamer constructs comprising the Fc domain variants that selectively bind FcγRIIB (e.g., as shown in FIG. 11; SEQ ID NOs: 7-8 and 12-13, preferably SEQ ID NO:7 or SEQ ID NO:8), and related methods of using such constructs, are also provided.

Some aspects of the present disclosure relate to a polypeptide comprising a mutant or variant human IgG Fc domain capable of binding human FcγRIIb, wherein the mutant or variant human IgG Fc domain comprises substitution mutations of valine at position 233 (E233V), leucine at position 239 (S239L), proline at position 238 (H268P), leucine at position 327 (A327L), alanine at position 328 (L328A), and substitution mutations at positions 234 (L234) and 235 (L235); with amino acid position numbering being according to the Kabat system. The mutant or variant human IgG Fc domain may preferably further comprise substitution mutations of glycine at position 298 (S298G) and alanine at position 299 (T299A). The substitution mutation at position 234 may be proline at position 234 (L234P) or aspartic acid at position 234 (L234D). In some embodiments, the substitution mutation at position 234 is aspartic acid at position 234 (L234D). The substitution mutation at position 235 may be threonine at position 235 (L235T) or phenylalanine at position 235 (L235F). In some embodiments, the substitution mutation at position 235 is phenylalanine at position 235 (L235F). In some embodiments, the substitution mutation at position 234 is proline at position 234 (L234P), and wherein the substitution mutation at position 235 is threonine at position 235 (L235T). The mutant or variant human IgG Fc domain may further comprises a substitution mutation of aspartic acid at position 237 (S267D). In some embodiments, the mutant or variant human IgG Fc domain further comprises a substitution mutations of glutamine at position 332 (I332Q) and/or valine at position 334 (K334V). In some embodiments, the mutant or variant human IgG Fc domain comprises or consists of Fc V8.2 (SEQ ID NO:2). In some embodiments, the substitution mutation at position 234 is aspartic acid at position 234 (L234D), and wherein the substitution mutation at position 235 is phenylalanine at position 235 (L235F). The mutant or variant human IgG Fc domain may further comprise 1, 2, 3, or all of: substitution mutations arginine at position 236 (G236R), aspartic acid at position 237 (G237D), histidine at position 330 (A330H), and/or isoleucine at position 333 (E333I). The mutant or variant human IgG Fc domain may further comprise a substitution mutation of aspartic acid at position 267 (S267D). In some embodiments, the mutant or variant human IgG Fc domain comprises or consists of Fc 2B18K (SEQ ID NO:3). The mutant or variant human IgG Fc domain may further comprise a substitution mutation of glutamine at position 292 (R292Q). The mutant or variant human IgG Fc domain may comprise or consists of Fc 2B18KQ (SEQ ID NO:4). The mutant or variant human IgG Fc domain may further comprise a substitution mutation of glutamine at position 292 (R292Q). The mutant or variant human IgG Fc domain may comprise or consist of Fc 2B18KQS (SEQ ID NO:5). In some embodiments, the mutant or variant human IgG Fc domain is aglycosylated. In some emb n some embodiments, the mutant or variant human IgG Fc domain is glycosylated. In some embodiments, the Fc domain does not selectively or detectably bind to 1, 2, 3, 4, or all of: a human FcγRI, FcγRIIa H131, FcγRIIIa F158, FcγRIIIa V158, and/or C1q polypeptide. In some embodiments, the Fc domain does not selectively or detectably bind to a human FcγRI. In some embodiments, the Fc domain does not selectively or detectably bind to any of human FcγRIIa H131, FcγRIIIa F158, and FcγRIIIa V158. In some embodiments, the Fc domain has a binding to FcγRIIa_{R131} that is at least 30-fold or 40-fold less than wild-type Fc binding. The Fc domain may further comprise a substitution mutation at position 299 (e.g., a leucine at amino acid position 299 (T299L)) and/or a substitution mutation at position 298. In some embodiments, the Fc domain comprises serine at position 298 and threonine position 299. The polypeptide may further comprise a non-Fc receptor (non-FcR) binding domain. The non-FcR binding domain may be an Ig variable domain, an antibody variable domain, or an antibody heavy chain variable domain. In some embodiments, the polypeptide is a full-length antibody or an agonistic antibody (e.g., an anti-CD40 agonist antibody). The Ig variable domain may comprise an antibody heavy chain variable domain. The Ig variable domain may be comprised in a single domain antibody. The Ig variable domain may comprise an ScFv. The mutant or variant human IgG Fc domain may be comprised in a multimeric oligomer (e.g., a hexameric Fc fusion protein). The hexameric Fc fusion protein may comprises PTLYNVSLVMSDTAGTCY (SEQ ID NO:6), PTLYNVSLIMSDTGGTCY (SEQ ID NO:9), PTLYNVSLIMSDTAGTCY (SEQ ID NO:10), or PTLYNVSLVMSDTGGTCY (SEQ ID NO:11). As shown in the below examples, these peptides can be used to promote formation of hexameric Fc fusion proteins. In some preferred embodiments, the hexameric Fc fusion protein comprises PTLYNVSLVMSDTAGTCY (SEQ ID NO:6). The mutant or variant human IgG Fc domain may comprise a substitution mutation of Leu residue at position 309, with amino acid position numbering being according to the Kabat system. The hexameric Fc fusion protein may be glycosylated or aglycosylated. In some embodiments, the mutant or variant human IgG Fc domain comprises a Gly residue at position 298 and/or an Ala residue at position 299. In some embodiments, the mutant or variant human IgG Fc domain comprises a Ser residue at position 298 and/or a Thr residue at position 299. In some preferred embodiments, the hexameric Fc fusion protein comprises SEQ ID NO:7, SEQ ID NO:8, SEQ ID NO:12, or SEQ ID NO:13, and the the hexameric Fc fusion protein may even more preferably comprises SEQ ID NO:7 or SEQ ID NO:8. The polypeptide or antibody may be chemically conjugated to or covalently bound to a toxin. The non-FcR binding region may bind a cell-surface protein. In some embodiments, the non-FcR binding region binds a soluble protein. The non-FcR binding domain may comprise a single domain antibody, a scFv, or a nanobody. The polypeptide may be aglycosylated or glycosylated. In some embodiments, the Fc domain triggers no or essentially no antibody mediated phagocytosis. In some embodiments, the Fc domain triggers no or essentially no antibody mediated cell cytotoxicity. In some embodiments, the Fc domain causes no or essentially no induction of activating FcγR.

Another aspect of the present disclosure relates to a nucleic acid encoding any of the polypeptides described above or herein. The nucleic acid may be a DNA segment. In some embodiments, the nucleic acid is an expression vector.

Yet another aspect of the present disclosure relates to a host cell comprising the nucleic acid described herein of above. The host cell may express the nucleic acid. The host cell may be a eukaryotic cell. In some embodiments, the host cell is a mammalian cell, an insect cell, or a yeast cell.

Another aspect of the present disclosure relates to a method for preparing an aglycosylated polypeptide comprising: a) obtaining a host cell as described above or herein; b) incubating the host cell in culture under conditions to promote expression of the aglycosylated polypeptide; and c) purifying the expressed polypeptide from the host cell. The host cell may be a eukaryotic cell (e.g., a mammalian cell, insect cell, or a yeast cell).

Yet another aspect of the present disclosure relates to a pharmaceutical formulation comprising a polypeptide described above or herein, or the nucleic acid described above or herein in a pharmaceutically acceptable carrier.

Another aspect of the present disclosure relates to a method of binding a protein in a mammalian subject comprising administering to the subject an antibody, wherein the antibody is binds the protein and comprises an Fc domain described above or herein. In some embodiments, the antibody is capable of specifically binding human FcγRIIb, and wherein the antibody has a reduced binding of one or more activating Fcγ receptors as compared to a human wild-type IgG Fc domain. The antibody may be aglycosylated or glycosylated. In some embodiments, the antibody results in no or essentially no antibody-mediated phagocytosis in the subject after the administering. In some embodiments, the mammalian subject is a human. In some embodiments, the antibody binds FcγRIIa_{R131} receptor in the subject with an affinity that is at least about 30-fold less than or about 40-fold less than a wild-type Fc. In some embodiments, the antibody does not selectively or detectably bind one or more activating human Fcγ receptor polypeptide in the subject. The activating human Fcγ receptor polypeptide may be FcγRI, FcγRIIa H131, FcγRIIIa F158, and/or FcγRIIIa V158. In some embodiments, the antibody does not specifically or detectably bind one or more activating human C1q. The antibody may be an aglycosylated version of a therapeutic antibody.

Yet another aspect of the present disclosure relates to a method of treating a subject having a disease comprising administering to the subject an effective amount of the formulation described above or herein. In some embodiments, the method does not induce antibody-dependent cytotoxicity. The disease may be a cancer, an infection, or an autoimmune disease. In some embodiments, the subject is a human patient.

In some embodiments, mutant or variant Fc domains are provided that, as compared to a corresponding wild-type Fc domain, exhibit: selective binding for FcγRIIB and (ii) reduced binding or no detectable binding to all the activating human Fcγ receptors namely FcγRI, FcγRIIa H131, FcγRIIa R131, FcγRIIIa F158, or FcγRIIIa V158) and C1q. In some preferred embodiments, the mutant or variant Fc domains are human mutant or variant Fc domains. The mutant or variant Fc domain may be comprised in or included in a polypeptide, such as an antibody or a fusion protein. In some embodiments, the mutant or variant Fc domain may be comprised in a therapeutic antibody such as, e.g., an agonistic antibody. In some embodiments, there are compositions involving a polypeptide that has an aglycosylated Fc domain from a human IgG1 antibody (“antibody Fc domain”). In some embodiments, the Fc domain is a variant of the human IgG1 Fc domain (SEQ ID NO: 1) that enables highly selective binding only to FcγRIIB and not to any of the effector Fc receptors namely FcγRI, FcγRIIA, and FcγRIIIA. The Fc domain may bind highly selectively to FcγRIIB and displays little or no binding to effector Fc receptors, both when it is expressed in glycosylated or aglycosylated form.

Another aspect of the present invention relates to a pharmaceutically acceptable composition comprising a polypeptide of the present invention and a pharmaceutically acceptable excipient.

Yet another aspect of the present invention relates to a composition for use in a method of treating a disease in a subject in need thereof, said composition comprising a polypeptide of the present disclosure. In some embodiments, said disease is a cancer, an infection, a bacterial infection, a viral infection, or an autoimmune disease.

An antibody Fc domain may be the Fc domain of an IgG antibody or a variant thereof. Furthermore, the antibody Fc domain may be defined as a human Fc domain. In certain aspects, the Fc domain may be an IgG1 Fc domain, such as the Fc domain of an anti-HER2 antibody, more specifically, the Fc domain of trastuzumab and the Fc domain of an anti-CD20 antibody, more specifically, the Fc domain of rituximab. It is also contemplated that a polypeptide may comprise a fusion of an engineered Fc domain as disclosed herein fused to a polypeptide not derived from an antibody molecule.

In some cases, the antibody may also have a substitution at amino acid 297 or 299 that impairs N-linked glycosylation when the antibody is expressed in mammalian cells that recognize the glycosylation motif in the antibody Fc domain. It is anticipated that any mutation in amino acid 297 or 299 known to abolish glycosylation can be used (e.g., WO2005018572A2) can be employed including, e.g., replacement of 299T by a leucine residue.

In some embodiments there are multiple amino acid substitutions at one or more positions from the following list: (264, 328, 329, 330, 332, 333), (336; 234, 235, 236, 238), and/or (351 and 311); in some embodiments, the engineered Fc domain may have a substitution mutation at 2, 3, 4, 5, 6, 7, 8, 9, 10, 11, 12, or all of these positions. An aglycosylated antibody Fc domain described herein may comprise a substitution at amino acid 264 to alanine (V264A), a substitution at amino acid 328 by serine (L328S), a substitution at amino acid 329 to cysteine (P329C), a substitution at amino acid 330 to tryptophan (A330W), a substitution at amino acid 332 to asparagine (I332N), a substitution at amino acid 333 to glycine (E333G), a substitution at amino acid 336 to valine (I336V) or combinations of these substitutions thereof. The engineered Fc domain may further comprise one or more substitution(s) at amino acids 234, 235, 236, 238, and 351; and in some embodiments, the substitution at amino acid 234 is arginine (L234R), the substitution at amino acid 235 is glutamate (L235E), the substitution at amino acid 236 is glutamate (G236E), the substitution at amino acid 238 is arginine (P238R), and the substitution at amino acid 351 is glutamine (L351Q). In some embodiments, the engineered Fc domain contains an additional amino acid substitutions at residue 311 such as, e.g., lysine (i.e., Q311K) in some preferred embodiments. Combinations of substitution mutations may be present in a mutant or variant Fc domain of the present disclosure. The mutant or variant human IgG Fc domain may comprise 1, 2, 3, or 4 of: substitution mutations of alanine at amino acid 264 (V264A), cysteine at amino acid 329 (P329C), glycine at position 333 (E333G), and valine at amino acid position 336 (I336V); optionally in combination with 1, 2, or 3 of: tryptophan at amino acid 330 (A330W), asparagine at amino acid 332 (1332N), and serine at amino acid 328 (L328S); optionally in combination with threonine at position 299 (T299L). In some embodiments, the variant Fc domain may comprise 1, 2, 3, 4, 5, 6, or all of: V264A, L328S, P329C, A330W, I332N, E333G, I336V mutations, optionally in combination with a mutation at position 299 such as T299L. In some embodiments, the variant Fc domain may comprise 1, 2, 3, 4, 5, or all of: L234R, L235E, G236E, P238R, T299L, and/or L351Q mutations. In some embodiments, the variant Fc domain comprises the Q311K mutation, optionally in combination with the T299L mutation. An engineered antibody Fc domain described herein may further comprise one or more amino acid substitutions disclosed in U.S. Pat. No. 10,526,408.

A variant Fc domain polypeptide (also referred to as a mutant or engineered Fc domain) may be characterized as having a certain percentage of identity as compared to an unmodified polypeptide (e.g., a wild-type Fc domain polypeptide, such as a wild-type IgG Fc domain, or a human wild-type IgG Fc domain) or to any polypeptide sequence disclosed herein. The percentage identity may be about, at least 70%, 75%, 80%, 85%, 90%, 95%, 96%, 97%, 98%, 99%, 99.5%, or 100% (or any range derivable therein) between the unmodified portions of a modified polypeptide (i.e., the sequence of the modified polypeptide excluding any specified substitutions) and the corresponding wild-type polypeptide. For example, a variant Fc domain may have, e.g., at least 90% (or at least about 95%, etc.) sequence identity as compared to a wild-type Fc domain (e.g., a wild-type human Fc domain) for regions of the variant Fc domain excluding specified substitution mutations (e.g., a substitution mutation at position 299 (e.g., T299L), in addition to any other specified substitution mutation(s)). The variant Fc domain may contain additional mutations, as compared to a wild-type Fc domain, in addition to the specified substitution mutations in the mutant Fc domain. It is also contemplated that percentage of identity discussed above may relate to the entirety of a variant Fc domain polypeptide as compared to a wild-type Fc domain (e.g., a human IgG Fc domain). For example, a variant Fc domain polypeptide characterized as having at least 90% identity to a wild-type Fc domain means that at least 90% of the amino acids in that variant polypeptide are identical to the amino acids in the wild-type polypeptide.

An antibody Fc domain may be an Fc domain of a human IgG antibody or a variant thereof. In certain aspects, the Fc domain may be an IgG1 Fc domain. It is also contemplated that a polypeptide may comprise a fusion of an engineered variant Fc domain as disclosed herein fused to a polypeptide not derived from an antibody molecule. In some embodiments, an engineered Fc domain of the present invention is comprised in an agonistic antibody such as, e.g., an antibody targeting CD40, death receptor 5 (DR5), or a TNF receptor (TNFR) molecule.

Polypeptides comprising a variant Fc domain described herein may include a linker in some embodiments. In further embodiments, the linker is a conjugatable linker. In some embodiments, the polypeptide contains an Fc domain from an antibody. It may contain other regions from an antibody, such as another binding domain. The additional binding domain may not be an FcR binding domain. In some embodiments, the polypeptide may contain an antigen binding site or domain from an antibody, such as all or part of the variable region from an antibody. The polypeptide may contain an Fc domain from an antibody and another binding domain that is a non-FcR binding domain. In some embodiments, the non-Fc binding region is not an antigen binding site of an antibody but specifically binds a cell-surface protein or a soluble protein. In some cases, a cell-surface protein that the non-Fc binding region recognizes is a receptor, such as, e.g., a receptor expressed on a cell surface.

Other polypeptides include those having an aglycosylated variant Fc domain (e.g., capable of binding a FcγRIIb polypeptide while exhibiting reduced or eliminated binding to an activating FcR) and a second binding domain that is a non-Fc receptor binding domain, wherein the second binding domain is capable of specifically binding a cell-surface molecule or a soluble protein. In some embodiments, the second binding domain is an antigen binding domain of an antibody (“Ig variable domain”). In some aspects, the polypeptide may be a full-length antibody. In some cases, the second binding domain is not an antibody antigen binding domain. In some embodiments, the second binding domain is capable of specifically binding a cell-surface molecule that is a protein or proteinaceous molecule. In some aspects, the second binding domain is capable of specifically binding a soluble protein.

Some aspects concern a nucleic acid that encodes any of the polypeptides discussed herein. The nucleic acid may be isolated and/or recombinant. It may be a nucleic acid segment that is isolated and/or recombinant. In some embodiments, the nucleic acid is DNA, while in others it is RNA. In some embodiments, the nucleic acid is a DNA segment. In some embodiments, the nucleic acid is an expression vector that is capable of expressing any of the polypeptides having an Fc binding domain with one or more substitutions that specifically binds FcγRIIb. A nucleic acid may encode one or more polypeptides herein, which, depending on the presence or absence of certain mutations, as well as how the polypeptide is produced, may or may not be glycosylated.

In some embodiments, the nucleic acid encodes a polypeptide comprising or consisting of a variant or mutant Fc domain capable of selectively binding FcγRIIb as described herein. The nucleic acid may be placed (e.g., transfected or transformed) into a host cell that can express the polypeptide, such as an aglycosylated version of the polypeptide. The host cell may be a prokaryotic cell, such as a bacterial cell. Alternatively, the host cell may be a eukaryotic cell, such as a mammalian cell. In some embodiments, a host cell contains a first expression vector, though it may also comprise a second expression vector as well. Because some antibodies are made of multiple polypeptides, a host cell that contains the expression vector(s) needed to express the polypeptides may be utilized in some embodiments. For example, in some embodiments the host cell includes a second expression vector that encodes a polypeptide comprising or consisting of an immunoglobulin light chain. In some embodiments, the host cell expresses a first expression vector encoding a polypeptide comprising or consisting of an immunoglobulin heavy chain (e.g., containing a variant or mutant Fc domain that selectively binds FcγRIIb). The host cell may comprise, e.g., one or two expression vectors to allow for the expression of an antibody comprising a heavy chain and a light chain.

In some aspects, a population of host cells is provided, wherein the population contains a plurality of host cells that express polypeptides having different Fc domains. It is contemplated that the amino acid sequence of any two different Fc domains may differ in identity by less than 20%, 15%, 10%, 5%, or less.

In some aspects, provided are methods of making the polypeptides described herein (e.g., polypeptides having an aglycosylated Fc region that can selectively bind FcγRIIb) and methods of using these polypeptides. It is anticipated that methods described herein or known to one of ordinary skill may be to generate or use any of the polypeptides described herein.

In some embodiments, there are methods for preparing an aglycosylated polypeptide comprising: a) obtaining a host cell capable of expressing an aglycosylated polypeptide comprising an Fc domain capable of selectively binding FcγRIIb as described herein; b) incubating the host cell in culture under conditions to promote expression of the aglycosylated polypeptide; and, c) purifying expressed polypeptide from the host cell. In some embodiments, the host cell is a prokaryotic cell, such as a bacterial cell. In other embodiments the host cell is a eukaryotic cell and the polypeptide comprises a substitution mutation at position 299 (e.g., T299L) of the variant or mutant IgG Fc domain. In further embodiments, methods involve collecting the expressed variant polypeptide (e.g., from the supernatant), which may be done prior to purification.

In some embodiments, methods involve purifying the polypeptide from the supernatant. This may involve subjecting the polypeptides from the supernatant to filtration, HPLC, anion or cation exchange, high performance liquid chromatography (HPLC), affinity chromatography or a combination thereof. In some embodiments, methods involve affinity chromatography using staphylococcal Protein A, which binds the IgG Fc region. Other purification methods are well known to those of ordinary skill in the art.

In some embodiments, there is provided a pharmaceutical formulation comprising a polypeptide or nucleic acid of the present disclosure in a pharmaceutically acceptable carrier or a pharmaceutical preparation comprising an excipient.

In some embodiments, an immune response may be induced in a subject by a method comprising providing or administering (e.g., intravenously, etc.) to the subject an antibody, wherein the antibody is aglycosylated and comprises an Fc domain that selectively binds FcγRIIb, as described herein. In some aspects, the aglycosylated antibody may be capable of specifically binding human FcγRIIb. In some aspects, the aglycosylated antibody may be capable of specifically binding any of the activating FcγR polypeptides at a level that is at least 30-fold or 40-fold lower than wild-type human IgG1 antibodies. In some embodiments, the antibody may comprise a mutant Fc domain provided herein that exhibits no specific or detectable binding to an FcγRI polypeptide, does not stimulate antibody-mediated phagocytosis, and/or does not trigger effector functions in a mammalian host. In some aspects, the antibody may be a glycosylated or aglycosylated version of a therapeutic antibody.

In some aspects, cancer, infection, autoimmune or inflammatory diseases may be treated by administering a therapeutic polypeptide comprising a variant or mutant Fc domain that selectively binds FcγRIIb as described herein. The polypeptide comprising a mutant or variant Fc domain as described herein may exhibit a decreased CDC compared to the CDC induced by a polypeptide comprising a wild-type human IgG Fc region. The polypeptide comprising a mutant Fc domain provided herein may exhibit reduced ADCC or ADCP as compared to wild-type human IgG antibodies.

In a further embodiment, therapeutic inhibition of a protein target may be achieved by antibodies comprising variant Fc polypeptides as contemplated herein. In some embodiments involving a polypeptide comprising a variant or mutant Fc domain that can selectively bind the inhibitory FcγRIIb while exhibiting decreased binding to activating Fc, the polypeptide may exhibit a reduced CDC compared to the CDC induced by a polypeptide comprising a wild-type human IgG Fc region.

Methods are provided for treating a subject having a disease comprising administering to the subject an effective amount of a pharmaceutical formulation of the present disclosure. In some embodiments, the tumor is a solid tumor or a hematological tumor. In certain aspects, the subject may be a human patient. In some aspects, the pharmaceutical formulation may be administered intratumorally, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctival, intravesicularlly, mucosally, intrapericardially, intraumbilically, orally, by inhalation, by injection, by infusion, by continuous infusion, by localized perfusion bathing target cells directly, via a catheter, or via a lavage. In some aspects, the method may further comprise administering at least a second anticancer therapy to the subject, such as, for example, a surgical therapy, chemotherapy, radiation therapy, cryotherapy, hormone therapy, immunotherapy, or cytokine therapy.

In one embodiment, a composition comprising a variant Fc domain of the present embodiments or a nucleic acid encoding a variant Fc domain of the present embodiments is provided for use in the treatment of a disease. Treating the disease may involve binding a select protein to achieve a therapeutic effect (e.g., resulting from binding of a toxin, or stimulation of a receptor with an agonistic antibody, etc.) while generating a reduced immune activation or reduced complement dependent cytotoxicity. In some aspects, the disease may be a cancer, an autoimmune disease, an inflammatory disease, or an infectious disease. In another embodiment, the use of a polypeptide according to the present embodiments or a nucleic acid encoding a polypeptide according to the present embodiments in the manufacture of a medicament for the treatment of a disease such as cancer is provided.

As used herein, “selectively binding FcγRIIb” or “selectively binds FcγRIIb” refer to a property of a polypeptide such as a Fc domain (e.g., a mutant or variant IgG Fc domain) to have the ability to bind FcγRIIb, and preferably the polypeptide or Fc domain has the ability to binding of FcγRIIb as compared to a wild-type Fc domain (e.g., a wild-type Fc IgG domain). The binding to the FcγRIIb may be comparable to or reduced (e.g., 4-fold reduced) as compared to the wild-type binding, but in all cases the mutant Fc domain must display a detectable selective binding of FcγRIIb. In some embodiments, a Fc domain or polypeptide that selectively binds FcγRIIb also displays either drastically reduced binding as compared to wild-type (e.g., a wild-type IgG Fc domain) or no detectable binding to all human activating (pro-inflammatory) Fcγ receptor In some embodiments, a mutant Fc domain provided herein binds FcRn with an affinity that is similar to or not significantly different from the wild-type Fc.

In some embodiments, a mutant or variant Fc domain provided above or herein is comprised in an an antibody or covalently attached to an antibody fragment (e.g., a heavy chain antibody variable domain, a scFv, etc.). In general, the term “antibody” includes, but is not limited to: polyclonal antibodies, monoclonal antibodies, single chain antibodies (containing a single heavy chain variable region, also called VHH), humanized antibodies, a deimmunized antibodies, minibodies, dibodies, tribodies as well as antibody fragments, such as Fab′, Fab, F(ab′)₂, single domain antibody, Fv, or single chain Fv (scFv) antibody single domain antibodies, and antibody mimetics, such as anticalins, and any mixture thereof. In some embodiments, single-domain antibodies (sdAbs) may allow certain advantages over full-length antibodies such as smaller size, larger number of accessible epitopes, and reduced production costs (e.g., Hoey et al., 2019). In some embodiments, the mutant of variant Fc domain is covalently attached to, or expressed as a fusion protein with, a single chain antibody (scFv) or a single domain antibody. In a related aspect, the cell targeting domain may be an avimer polypeptide.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein, the term “affinity” refers to the equilibrium constant for the reversible binding of two agents and is expressed as K_{D}. Affinity of a binding domain to its target can be, for example, from about 100 nanomolar (nM) to about 0.1 nM, from about 100 nM to about 1 picomolar (pM), or from about 100 nM to about 1 femtomolar (fM); alternatively, it can be between 100 nM and 1 nM or between 0.1 nM and 10 nM, or any range derivable therein. Moreover, it is contemplated that agents specifically bind when there is an affinity between the two agents that is in the affinity ranges discussed above.

As used herein the terms “encode” or “encoding,” with reference to a nucleic acid, are used to make the invention readily understandable by the skilled artisan; however, these terms may be used interchangeably with “comprise” or “comprising,” respectively.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1: Opsonized SK-BR-3 and FcγR coated beads binding assay result with 2b18K Fc, and 2b enhanced binding Fc variants. The numbers shown refer to the fold-difference of binding affinity compared to wild type Fc measured by BLI, and the numbers in the basket (parentheses shown in the figure) are published values. The table shows the percentage of the positive population and MFI (median fluorescence intensity) of the results. NA: not assayed; NB: no-binding.

FIG. 2: Opsonized SK-BR-3 and FcγR coated beads binding assay result with 2b18K Fc and silence Fc variants. The percentage of the positive population is shown in this graph. Error bars are standard errors of the mean of triplicate samples.

FIG. 3: Raji cell binding assay with antibody-coated beads. Statistical analysis was performed by one-way ANOVA with Tukey s multiple comparisons test (***P 0.001, ****P 0.0001). Error bars are standard errors of the mean of triplicate samples.

FIG. 4: Antibody opsonized pHrodo labeled beads phagocytosis assay with THP-1 cells as effectors. Statistical analysis was performed by one way ANOVA with Tukey s multiple comparisons test (***P 0.001). Error bars are standard errors of the mean of triplicate samples.

FIG. 5: SK-BR-3 phagocytosis assay with THP-1 cells. Numbers are the concentration of antibodies in media (ng/ml). ET ratio 20:1 was used. Error bars are standard errors of the mean of triplicate samples. non: isotype control. Statistical analysis was performed using two-way ANOVA with Tukey's multiple comparisons test (****P 0.0001).

FIG. 6: SK-BR-3 phagocytosis assay with THP-1 cells. Numbers are shown at the bottom of the figure and refer to the concentration of antibody in media (ng/ml). ET ratio 20:1 was used. Error bars are standard error of the mean of triplicate samples. “non” refers to isotype control.

FIG. 7: Alignment of amino acid sequences.

FIG. 8: Deposition of C1q on CD20+ Raji or Ramos cells as measured by FACS. Cells were opsonized using WT rituximab or Fc-engineered variants. Fc variants showed low or no C1q deposition on the cell surface. Data for Fc mutants 2b18K (Fc2b-1), 2b18KQ (Fc2b-2), and 2b18KQS (Fc2b-3) are shown.

FIGS. 9A-E: In vitro cell-based assays. Data for Fc mutants 2b18K (Fc2b-1), 2b18KQ (Fc2b-2), and 2b18KQS (Fc2b-3) are shown. No complement or activating FcγR-mediated effector functions were detected using Fc variants 2b18K, 2b18KQ, or 2b18KQS. (FIG. 9A) Lysis of CD20+ Raji cells or Ramos cells by complement-dependent cytotoxicity (CDC). Cells were opsonized by various concentrations of rituximab or its Fc-engineered variants and incubated with 10% pooled human serum. (FIG. 9B) Antibody-dependent cellular phagocytosis assay (ADCP) with THP-1 cells as effectors and SK-BR-3 cells as targets were performed using WT or Fc-engineered variants of trastuzumab. (FIG. 9C) Antibody-dependent cellular cytotoxicity assay (ADCC) with peripheral blood mononuclear cells (PBMCs). The target cells were Raji cells opsonized with WT rituximab or its Fc-engineered variants. (FIG. 9D) ADCP assay with human monocyte-derived M1 macrophages as effectors and SK-BR-3 cells as targets. Various concentrations of WT trastuzumab or Fc-engineered variants were added. (FIG. 9E) Phosphorylation of FcγRIIb after incubating CD20+ Raji cells for 1 hour with WT rituximab or Fc-engineered variants analyzed by immunoblotting. In all assays, the Fc variants 2b18K, 2b18KQ, and 2b18KQS did not show any complement or activating FcγR-mediated activation, and these variants showed functional binding to FcγRIIb.

FIGS. 10A-D: Hexameric Fc expression. (FIG. 10A) Schematic figure of hexameric Fc. (FIG. 10B) reduced SDS-PAGE result of hexameric Fc. (FIG. 10C) non-reduced SDS-PAGE result of hexameric Fc after SEC purification. (FIG. 10D) Size exclusion chromatography results of hexameric Fc.

FIG. 11: Sequence alignment showing Hexameric wild-type Fc and point mutations present in the different engineered variant Fc regions. Hex-WT (SEQ ID NO:14) is shown and mutations in Hex-2bKQS and Hex-2bKQS, as compared to Hex-WT, are shown.

FIG. 12: Binding characteristics of Hexameric Fc2b. FcγR coated beads and hexameric Fc binding assay results with Hex 2b18KQS Fc and glycosylated Hex 2b18KQS-ST Fc are shown. The y-axis shows the mean fluorescence intensity.

FIG. 13: Raji cell binding assay with antibody coated beads. Error bars are standard error of the mean of triplicate samples. Statistical analysis was performed by one way ANOVA with Tukey s multiple comparisons test (***P 0.001, ****P 0.0001).

FIG. 14: Blocking FcgR2b with Hexameric Fc Increase of Antibody Opsonized SKBR3 Cell Phagocytosis by THP-1 cells. y-axis: percentage of ADCP, Error bars are standard error of the mean of triplicate samples.

### DESCRIPTION OF ILLUSTRATIVE EMBODIMENTS

Provided herein are methods and compositions involving polypeptides having engineered antibody Fc domains displaying selective binding to FcγRIIB, while exhibiting reduced or eliminated actions on activating FcγR (e.g., reduced or eliminated antibody-mediated phagocytosis, reduced or eliminated effector functions). Such polypeptides may comprise an Fc domain that comprises one or more substitution mutations as compared to a wild-type Fc domain (SEQ ID NO: 1). Additionally, some Fc domains may bind selectively to FcγRIIB but not activating FcγR (e.g., no detectable binding to FcγRIIaH131, FcγRIIIa, or FcγRIIIb). For example, polypeptides may comprise an aglycosylated Fc domain that selectively binds FcγRIIB, but that does not detectably bind to activating FcγRs. In some embodiments, the Fc domain displays decreased binding to FcγRIIaR131 (e.g., 40-fold lower binding as compared to WT Fc). Reduced or eliminated effector function induction by the mutant Fc may provide significant advantages for treating diseases where such an immune response (e.g., antibody-stimulated phagocytosis) would be undesirable.

### I. ANTIBODY Fc DOMAINS

FcγRIIB-bound Fc domain of IgG have been shown to suppress the activation of diverse immune cells in a variety of different assays (Sidman, C. L. and Unanue, E. R. 1976; Phillips, N. E. and Parker, D. C. 1984). FcγRIIB is the only FcγR expressed by B cells, and if it is cross-linked to the B cell receptor (BCR) the threshold for B cell activation is increased and B cell differentiation and eventually antibody production are decreased. In other immune cells, including dendritic cells (DCs), macrophages, activated neutrophils, mast cells, and basophils, FcγRIIB inhibits the functions mediated by activating FcγRs including phagocytosis and pro-inflammatory cytokine release. When expressed by follicular DCs (FDCs), FcγRIIB is important for trapping the antigen-containing immune complexes that are thought to be crucial for driving the germinal center response (Qin et al. 2000; Barrington 2002). The diversity of FcγRIIB expression and function underlies its importance in regulating defense against infection and susceptibility to autoimmune disease.

Importantly binding to FcγRIIB on effector and stromal cells has been shown to be critical for the agonistic function of TNFRS therapeutic antibodies (agonistic antibodies targeting key TNF receptor (TNFR) molecules). Many TNFRS agonistic antibodies including anti-CD40 or death receptor 5 (DR5) have been shown to be of key importance for immune regulation and activation. Signaling by agonistic antibodies to targets such as CD40 has been shown to depend on ligation of the Fc domain of the antibody by FcγRIIB expressed on neighboring cells in the microenvironment (Nimmerjahn et al. 2005; Wilson et al. 2011).

The FcγR binding sites on IgG1 have been determined by co-crystal structures of Fc fragments and the extracellular domains of FcγRs. The binding sites are generally located on the CH2 domain. The IgG1 lower hinge region (Leu234-Ser239) and Asp265-Ser267 segment in the CH2 domain have a key role in the interaction with all FcγRs (Christine Gaboriaud et al., 2003 and Jenny M. Woof et al., 2004).

The CH2 domain has one N-glycosylation site at Apn297 and the N-linked glycosylation at Asn297 bridges the gap between the two CH2 domains. This bridge maintains the proper conformation of CH2 domains for binding to FcγRs. On the other hand, the removal of glycan at Asn297 drastically increases the conformation of CH2 domains such that aglycosylated Fcs bind to FcγRs with significantly reduced affinity or not at all, thus significantly diminishing ADCC, ADCP and other biological effects mediated by the Fc:FcγR interaction (Borrok et al., 2012).

In light of the importance of FcγRIIB binding for the biological function of antibodies there have been extensive efforts to engineer IgG1 Fc domains that bind to this receptor with increased affinity and/or selectivity relative to other Fcγ receptors. These efforts have all involved the engineering of glycosylated IgG1 to bind with higher affinity to FcγRIIB since antibodies that lack the glycan at position 297 and hence they are aglycosylated do not exhibit any binding to FcγRIIB. Two IgG1 Fc variants with markedly increased binding to FcγRIIB have been reported: the so called “EF-Fc” variant developed by Xencor and the “V12-Fc” variant by Chugai (Chu et al., 2008; Mimoto et al., 2013; WO 2012/115241 A1) The EF-Fc variant contains two mutations: S267E and L328F. The V12-Fc variant has five mutations: E233D, G237D, H268D, P271G, and A330R. The EF variant was reported to have a 430-fold lower KD (equilibrium dissociation constant for FcγRIIB while the V-12 variant showed 64-fold greater affinity. However, the Fc domain was selective for FcγRIIB. Specifically, the EF Fc domain showed a similar affinity for FcγRI and FcγRIIA_{H131} relative to authentic (wild-type) human IgG1 Fc domain and significantly enhanced affinity for FcγRIIA_{R131}. V12-Fc variant was reported to have similar affinity for FcγRIIA_{R131} and a decreased affinity for FcγRI and FcγRIIA_{H131}, relative to the native IgG1 Fc domain.

In certain embodiments, there are compositions comprising a proteinaceous molecule that has been modified relative to a native or wild-type protein. In some embodiments that proteinaceous compound has been deleted of amino acid residues; in other embodiments, amino acid residues of the proteinaceous compound have been replaced; while in still further embodiments both deletions and replacements of amino acid residues in the proteinaceous compound have been made. Furthermore, a proteinaceous compound may include an amino acid molecule comprising more than one polypeptide entity. As used herein, a “proteinaceous molecule,” “proteinaceous composition,” “proteinaceous compound,” “proteinaceous chain,” or “proteinaceous material” generally refers, but is not limited to, a protein of greater than about 200 amino acids or the full-length endogenous sequence translated from a gene; a polypeptide of 100 amino acids or greater; and/or a peptide of 3 to 100 amino acids. All the “proteinaceous” terms described above may be used interchangeably herein; however, it is specifically contemplated that embodiments may be limited to a particular type of proteinaceous compound, such as a polypeptide. Furthermore, these terms may be applied to fusion proteins or protein conjugates as well. A protein may include more than one polypeptide. An IgG antibody, for example, has two heavy chain polypeptides and two light chain polypeptides, which are joined to each other through disulfide bonds.

As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide,” and “peptide” are used interchangeably herein.

As used herein, an “amino acid residue” refers to any amino acid, amino acid derivative, or amino acid mimic as would be known to one of ordinary skill in the art. In certain embodiments, the residues of the proteinaceous molecule are sequential, without any non-amino acid residue interrupting the sequence of amino acid residues. In other embodiments, the sequence may comprise one or more non-amino acid moieties. In particular embodiments, the sequence of residues of the proteinaceous molecule may be interrupted by one or more non-amino acid moieties.

As used herein a “distinct Fc domain” may be defined as a domain that differs from another Fc by as little as one amino acid. Methods for making a library of distinct antibody Fc domains or nucleic acids that encode antibodies are well known in the art. For example, in some cases Fc domains may be amplified by error prone PCR. Furthermore, in certain cases a plurality of antibody Fc domains may comprise a stretch (1, 2, 3, 4, 5, 6, 7, 8, 9, 10 or more) of amino acids that have been randomized. In certain cases, specific mutations may be engineered into Fc domains. For example, in some aspects, residues that are normally glycosylated in an antibody Fc domain may be mutated. Furthermore, in certain aspects, residues that are normally glycosylated (or adjacent residues) may be used as a site for an insertion of 1, 2, 3, 4, 5, 6, 7, 8, 9, 10 or more amino acids.

A polypeptide may comprise an aglycosylated antibody Fc domain capable of binding an FcR polypeptide. In some aspects, the aglycosylated Fc domain may be further defined as having a specific affinity for an FcR polypeptide under physiological conditions. For instance an Fc domain may have an equilibrium dissociation constant between about 10−6 M to about 10−9 M under physiological conditions. Furthermore, in some aspects an aglycosylated Fc domain may be defined as comprising one or more amino acid substitutions or insertions relative to a wild-type sequence, such as a human wild-type sequence.

Means of preparing such a polypeptide include those discussed in PCT Publn. WO 2008/137475, which is hereby incorporated by reference. One can alternatively prepare such polypeptides directly by genetic engineering techniques such as, for example, by introducing selected amino acid substitutions or insertions into a known Fc background, wherein the insertion or substitution provides an improved FcR binding capability to aglycosylated Fc regions, as discussed above. In some embodiments, an Fc domain is engineered to bind one or more specific Fc receptors. Additionally, or alternatively, an Fc domain may be engineered so that it does not selectively bind one or more specific Fc receptors.

In some embodiments, an aglycosylated Fc domain comprises a specific binding affinity for an FcR such as human FcγRIA, FcγRIIA, FcγRIIB, FcγRIIc, FcγRIIIA, FcγRIIIb, FcαRI, or for C1q. Thus, in some aspects an aglycosylated Fc domain of the invention is defined as an Fc domain with a specific affinity for FcγRIIB. The binding affinity of an antibody Fc or other binding protein can, for example, be determined by the Scatchard analysis of Munson and Pollard (1980). Alternatively, binding affinity can be determined by surface plasmon resonance or any other well known method for determining the kinetics and equilibrium constants for protein:protein interactions.

Amino acids sequences of Fc domains of the isolated IgG variants with specific affinity for FcγRIIB with changes shown relative to wild-type Fc (SEQ ID NO: 1) are as follows:

TABLE 1
Isolated IgG variants with affinity for FcγRIIB (sequence
numbering is based on Kabat and mutations are specified below)

| Variant | Mutations |
|---|---|
| WT | — |
| V8.2 | E233V, L234P, L235T, S239L, S298G, S267D, H268P, |
| | T299A, A327L, L328A, I332Q, K334V |
| 2b18K | E233V, L234D, L235F, G236R, G237D, S239L, S267D, |
| | H268P, S298G, T299A, A327L, L328A, A330H, E333I |
| 2b18KQ | E233V, L234D, L235F, G236R, G237D, S239L, S267D, |
| | H268P, R292Q, S298G, T299A, A327L, L328A, A330H, |
| | E333I |
| 2b18KQS | E233V, L234D, L235F, G236R, G237D, S239L, H268P, |
| | R292Q, S298G, T299A, A327L, L328A, A330H, E333I |

Specific point mutations are listed for the mutant or variant Fc domains in Table 1 above; these mutations indicate differences between the mutant or variant Fc domain and a wild-type IgG Fc domain (SEQ ID NO:1). Some aspects of the present disclosure relate to a polypeptide having or a nucleic acid encoding an IgG Fc domain (such as an aglycosylated IgG Fc domain) having at least 85%, 90%, 95%, 96%, 97%, 98%, 99%, or 100%, or any range derivable therein, sequence identity to a mutant or variant Fc domain of Table 1. In some embodiments, a substitution mutation at T299 (e.g., T299L) is also included in a Fc mutant of Table 1, e.g., to allow for the production of an aglycosylated Fc domain in mammalian cells.

In some embodiments, the mutant of variant Fc domain comprises or consists of Fc 2B18K:

(SEQ ID NO: 3)
EPKSCDKTHTCPPCPAPVDFRDPLVFLFPPKPKDTLMISRTPEVTCVVV
DVDPEDPEVKFNWYVDGVEVHNAKTKPREEQYNGAYRVVSVLTVLHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

or a polypeptide having at least 85%, 90%, 95%, 96%, 97%, 98%, 99%, or 100%, or any range derivable therein, sequence identity.

In some embodiments, the mutant of variant Fc domain comprises or consists of Fc 2B18KQ:

(SEQ ID NO: 4)
EPKSCDKTHTCPPCPAPVDFRDPLVFLFPPKPKDTLMISRTPEVTCVVV
DVDPEDPEVKFNWYVDGVEVHNAKTKPQEEQYNGAYRVVSVLTVLHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

or a polypeptide having at least 85%, 90%, 95%, 96%, 97%, 98%, 99%, or 100%, or any range derivable therein, sequence identity.

In some embodiments, the mutant of variant Fc domain comprises or consists of Fc 2b18KQS:

(SEQ ID NO: 5)
EPKSCDKTHTCPPCPAPVDFRDPLVFLFPPKPKDTLMISRTPEVTCVVV
DVSPEDPEVKFNWYVDGVEVHNAKTKPQEEQYNGAYRVVSVLTVLHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

or a polypeptide having at least 85%, 90%, 95%, 96%, 97%, 98%, 99%, or 100%, or any range derivable therein, sequence identity.

In some embodiments, the mutant or variant Fc is comprised in a hexameric Fc polypeptide. For example, the mutant or variant Fc (e.g., SEQ ID NO:3, SEQ ID NO:4, or SEQ ID NO:5) may be comprised within a polypeptide that comprises a human IgM μ-tailpiece (e.g., PTLYNVSLVMSDTAGTCY; SEQ ID NO:6) that promotes polymerization of the Fc domains into a hexameric construct. For example, in some preferred embodiments the mutant of variant Fc domain (e.g., any one of SEQ ID NOs:3-6) is expressed as a fusion construct with the human IgM μ-tailpiece (e.g., SEQ ID NO:6) C-terminal to the mutant of variant Fc domain, or at the C-terminal end of the polypeptide.

It is anticipated that a variety of polypeptides can be included with a mutant or variant Fc as provided herein to produce a multimeric oligomer. In some preferred embodiments, a human IgM μ-tailpiece described in (Rowley et al., 2018; Spirig et al., 2018) is included to form a hexamer. The wherein mutant or variant human IgG Fc domain comprises a substitution mutation of Leu residue at position 309 according to Kabat numbering to introduce disulfide bonds between Fc domains and promote formation of a multimeric oligomer. The multimeric oligomer or hexamer may be glycosylated or aglycosylated. Other polypeptides that can be used to produce a multimeric oligomer (e.g., when expressed with a mutant of variant Fc provided herein) include PTLYNVSLIMSDTGGTCY (SEQ ID NO:9), PTLYNVSLIMSDTAGTCY (SEQ ID NO:10), or PTLYNVSLVMSDTGGTCY (SEQ ID NO:11) can be also used for hexameric formation, and the two mutations V567I and A572G may help stabilization of hexameric structures. (Yuan, et al., 2022)

In some embodiments, the hexameric Fc polypeptide comprises or consists of Hex 2b18KQS (aglycosylated Fc):

(SEQ ID NO: 7)
EPKSCDKTHTCPPCPAPVDFRDPLVFLFPPKPKDTLMISRTPEVTCVVV
DVSPEDPEVKFNWYVDGVEVHNAKTKPQEEQYNGAYRVVSVLTVCHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKPTLYNVSLVMSDT
AGTCY

or a polypeptide having at least 85%, 90%, 95%, 96%, 97%, 98%, 99%, or 100%, or any range derivable therein, sequence identity.

In some embodiments, the hexameric Fc polypeptide comprises or consists of Hex 2b18KQS-ST (glycosylated):

(SEQ ID NO: 8)
EPKSCDKTHTCPPCPAPVDFRDPLVFLFPPKPKDTLMISRTPEVTCVVV
DVSPEDPEVKFNWYVDGVEVHNAKTKPQEEQYNSTYRVVSVLTVCHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKPTLYNVSLVMSDT
AGTCY

or a polypeptide having at least 85%, 90%, 95%, 96%, 97%, 98%, 99%, or 100%, or any range derivable therein, sequence identity.

By “position” as used herein is meant a location in the sequence of a protein. Positions may be numbered sequentially, or according to an established format, for example the EU index for antibody numbering.

For all positions discussed herein, numbering is according to the EU index. The “EU index” or “EU index as in Kabat” or “Kabat numbering” or “EU numbering scheme” refers to the numbering of the EU antibody (Edelman et al., 1969; Kabat et al., 1991; both incorporated herein by reference in their entirety).

In certain embodiments the size of the at least one Fc polypeptide proteinaceous molecule may comprise, but is not limited to, about or at least 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 350, 400, 450, 500, 550, 600, 650, 700, 750, 800, 850, 900, 950, 1000 or greater amino molecule residues, and any range derivable therein. Compounds may include the above-mentioned number of contiguous amino acids from SEQ ID NO:1 (human IgG Fc polypeptide) or from a variant Fc domain as listed in Table 1 and these may be further qualified as having a percent identity or homology to SEQ ID NO: 1 (discussed herein).

A. Modified Proteins and Polypeptides

Some embodiments concern modified proteins and polypeptides, particularly a modified protein or polypeptide that exhibits at least one functional activity that is comparable to the unmodified version, yet the modified protein or polypeptide possesses an additional advantage over the unmodified version, such as suppressing B-cell activation, being easier or cheaper to produce, eliciting fewer side effects, and/or having better or longer efficacy or bioavailability. Thus, when the present application refers to the function or activity of a “modified protein” or a “modified polypeptide” one of ordinary skill in the art would understand that this includes, for example, a protein or polypeptide that 1) performs at least one of the same activities or has at least one of the same specificities as the unmodified protein or polypeptide, but that may have a different level of another activity or specificity; and 2) possesses an additional advantage over the unmodified protein or polypeptide. Determination of activity may be achieved using assays familiar to those of skill in the art, particularly with respect to the protein's activity, and may include for comparison purposes, for example, the use of native and/or recombinant versions of either the modified or unmodified protein or polypeptide. It is specifically contemplated that embodiments concerning a “modified protein” may be implemented with respect to a “modified polypeptide,” and vice versa. In addition to the modified proteins and polypeptides discussed herein, embodiments may involve domains, polypeptides, and proteins described in PCT Publn. WO 2008/137475, which is hereby specifically incorporated by reference.

Modified proteins may possess deletions and/or substitutions of amino acids; thus, a protein with a deletion, a protein with a substitution, and a protein with a deletion and a substitution are modified proteins. These modified proteins may further include insertions or added amino acids, such as with fusion proteins or proteins with linkers, for example. This may include the insertion of a targeting peptide or polypeptide or simply a single residue. Terminal additions, called fusion proteins, are discussed below.

A “modified deleted protein” lacks one or more residues of the native protein but possesses the specificity and/or activity of the native protein. A “modified deleted protein” may also have reduced immunogenicity or antigenicity. An example of a modified deleted protein is one that has an amino acid residue deleted from at least one antigenic region (i.e., a region of the protein determined to be antigenic in a particular organism, such as the type of organism that may be administered the modified protein).

Substitutional or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a native polypeptide are included, provided the biological activity of the protein is maintained. A modified protein may be biologically functionally equivalent to its native counterpart.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing of the amino acids of a protein to create an equivalent, or even an improved, second-generation molecule. For example, certain amino acids may be substituted for other amino acids in a protein structure with or without appreciable loss of interactive binding capacity with structures such as, for example, binding sites to substrate molecules. Since the interactive capacity and nature of a protein define that protein's biological functional activity, certain amino acid substitutions can be made in a protein sequence, and in its underlying DNA coding sequence, and nevertheless produce a protein with like properties. It is thus contemplated that various changes may be made in the DNA sequences of genes without appreciable loss of their biological utility or activity, as discussed below. A proteinaceous molecule has “homology” or is considered “homologous” to a second proteinaceous molecule if one of the following “homology criteria” is met: 1) at least 30% of the proteinaceous molecule has sequence identity at the same positions with the second proteinaceous molecule; 2) there is some sequence identity at the same positions with the second proteinaceous molecule and at the nonidentical residues, at least 30% of them are conservative differences, as described herein, with respect to the second proteinaceous molecule; or 3) at least 30% of the proteinaceous molecule has sequence identity with the second proteinaceous molecule, but with possible gaps of nonidentical residues between identical residues. As used herein, the term “homologous” may equally apply to a region of a proteinaceous molecule, instead of the entire molecule. If the term “homology” or “homologous” is qualified by a number, for example, “50% homology” or “50% homologous,” then the homology criteria, with respect to 1), 2), and 3), is adjusted from “at least 30%” to “at least 50%.” Thus, it is contemplated that there may homology or sequence identity of at least 30%, 35%, 40%, 45%, 50%, 55%, 60%, 65%, 70%, 75%, 80%, 85%, 90%, 95%, or more between two proteinaceous molecules or portions of proteinaceous molecules.

Alternatively, a modified polypeptide may be characterized as having a certain percentage of identity to an unmodified polypeptide or to any polypeptide sequence disclosed herein, including a mutant of variant Fc domain listed in Table 1. The percentage identity may be at most or at least 50%, 55%, 60%, 65%, 70%, 75%, 80%, 85%, 90%, 95%, 96%, 97%, 98%, 99% or 100% (or any range derivable therein) between two proteinaceous molecules or portions of proteinaceous molecules. It is contemplated that percentage of identity discussed above may relate to a particular region of a polypeptide compared to an unmodified region of a polypeptide. For instance, a polypeptide may contain a modified or mutant Fc domain that can be characterized based on the identity of the amino acid sequence of the modified or mutant Fc domain to an unmodified or mutant Fc domain from the same species. A modified or mutant human Fc domain characterized, for example, as having 90% identity to an unmodified Fc domain means that 90% of the amino acids in that domain are identical to the amino acids in the unmodified human Fc domain (SEQ ID NO:1).

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions typically are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine, and isoleucine.

B. Modified Antibodies and Proteinaceous Compounds with Heterologous Regions

Once an Fc domain has been isolated, it may be desired to link the molecule to at least one agent to form a conjugate to enhance the utility of that molecule. For example, in order to increase the efficacy of Fc domains or antibody molecules as diagnostic or therapeutic agents, it is conventional to link or covalently bind or complex at least one desired molecule or moiety. Such a molecule or moiety may be, but is not limited to, at least one effector or reporter molecule. Effecter molecules comprise molecules having a desired activity, e.g., cytotoxic activity. Non-limiting examples of effector molecules that have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands, such as biotin. Another such example is the formation of a conjugate comprising an antibody linked to a cytotoxic or anti-cellular agent, and may be termed “immunotoxins.” Techniques for labeling such a molecule are known to those of skill in the art and have been described herein above.

Labeled proteins, such as Fc domains that have been prepared in accordance with the present disclosure may also then be employed, for example, in immunodetection methods for binding, purifying, removing, quantifying, and/or otherwise generally detecting biological components, such as protein(s), polypeptide(s), or peptide(s). Some immunodetection methods include enzyme-linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature including, e.g., Doolittle and Ben-Zeev, 1999; Gulbis and Galand, 1993; and De Jager et al., 1993, each incorporated herein by reference.

The Fc domain molecules, including antibodies, may be used, for example, in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Abbondanzo et al., 1990).

Some embodiments concern an Fc polypeptide proteinaceous compound that may include amino acid sequences from more than one naturally occurring or native polypeptides or proteins. Embodiments discussed above are contemplated to apply to this section, and vice versa. For instance, a modified antibody is one that contains a modified Fc domain with an antigen binding domain. Moreover, the antibody may have two different antigen binding regions, such as a different region on each of the two heavy chains. Alternatively or additionally, in some embodiments, there are polypeptides comprising multiple heterologous peptides and/or polypeptides (“heterologous” meaning they are not derived from the same polypeptide). A proteinaceous compound or molecule, for example, could include a modified Fc domain with a protein binding region that is not from an antibody. In some embodiments, there are polypeptides comprising a modified Fc domain with a protein binding region that binds a cell-surface receptor. These proteinaceous molecules comprising multiple functional domains may be two or more domains chemically conjugated to one another or it may be a fusion protein of two or more polypeptides encoded by the same nucleic acid molecule. It is contemplated that proteins or polypeptides may include all or part of two or more heterologous polypeptides.

Thus, a multipolypeptide proteinaceous compound may be comprised of all or part of a first polypeptide and all or part of a second polypeptide, a third polypeptide, a fourth polypeptide, a fifth polypeptide, a sixth polypeptide, a seventh polypeptide, an eight polypeptide, a ninth polypeptide, a tenth polypeptide, or more polypeptides.

Amino acids, such as selectively-cleavable linkers, synthetic linkers, or other amino acid sequences, may be used to separate proteinaceous moieties.

Polypeptides or proteins (including antibodies) having an antigen binding domain or region of an antibody and an aglycosylated Fc domain can be used against any antigen or epitope, including but not limited to proteins, subunits, domains, motifs, and/or epitopes belonging to the following list of targets: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/ZIP, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MM), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, I-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors. In some embodiments, a polypeptide or protein has an antigen binding domain specific for one or more cell surface tumor antigens or B-cell antigen. Methods and compositions may be employed to target a tumor cell or B-cell.

Any antibody of sufficient selectivity, specificity, or affinity may be employed as the basis for an antibody conjugate. Such properties may be evaluated using conventional immunological screening methodology known to those of skill in the art. Sites for binding to biologically active molecules in the antibody molecule, in addition to the canonical antigen binding sites, include sites that reside in the variable domain that can bind pathogens, B-cell superantigens, the T cell co-receptor CD4, and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Fc domains can bind to an FcR, however, it is contemplated that the regulation of immune response can be directed not only through an antigen binding domain on the polypeptide containing the Fc domain, but through some other protein binding domain. Consequently, some embodiments may concern an Fc domain and a heterologous non-antigen binding domain. In certain embodiments, the non-antigen binding domain binds to the cell surface. Therefore, these agents require either chemical conjugation to, or fusion with, agents/proteins that are capable of binding to specific target cells. Embodiments may further include adjoining all or part of an aglycosylated Fc domain to all or part of any of the proteins listed in Table 2. It is contemplated that embodiments include, but are not limited to, the examples provided in Table 2 and the description herein.

A ligand for a receptor may be employed to target a cell expressing on its surface the receptor for the ligand. Ligands also include, for instance, CD95 ligand, TRAIL, TNF (such as TNF-α or TNF-β), growth factors, including those discussed above, such as VEGF, and cytokines, such as interferons or interleukins, and variants thereof. Embodiments with multiple domains are also contemplated, such as a VEGF Trap fusion protein that includes the second extracellular domain of the VEGF receptor 1 (Flt-1) with the third domain of the VEGF receptor 2 (KDR/FIK-1) and an IgG Fc region.

TABLE 2
Agents/proteins capable of binding specific target cells

| Protein | | | |
|---|---|---|---|
| Genus | Subgenus | Species | Subspecies |
| Antibodies | Polyclonal | | |
| | Monoclonal | Non-recombinant | |
| | | Recombinant | |
| | | | Chimeric |
| | | | Single chain |
| | | | Diabody |
| | | | Multimeric |
| Ligands | | | IL-1, IL-2, IL-3, |
| for cell- | | | IL-4, IL-5, IL-6, |
| surface | | | IL-7, IL-8, IL-9, |
| receptors | | | IL-10, IL-11, |
| | | | IL-12, IL-13, |
| | | | IL-14, IL-15, |
| | | | IL-16, IL-17, |
| | | | IL-18, IL-19 |
| | Cytokines/ | | |
| | growth factors | | |
| | | Cytokines/growth | |
| | | factors for receptor | |
| | | tyrosine kinases | |
| | | | GM-CSF, G-CSF, |
| | | | M-CSF, EGF, |
| | | | VEGF, FGF, PDGF, |
| | | | HGF, GDNF, Trk, |
| | | | AXL, LTK, TIE, |
| | | | ROR, DDR, KLG, |
| | | | RYK, MuSK ligands |
| Non-Ab | | | |
| binding | | | |
| protein | | | |
| for cell- | | | |
| surface | | | |
| molecule | | | |
| | Binders of cell | | |
| | surface proteins | | |
| | | Cluster of | |
| | | differentiation (CD) | |
| | | molecules | |

C. Antibody Fc Libraries

Examples of techniques that could be employed in conjunction with embodiments for creation of diverse antibody Fc domains and/or antibodies comprising such domains may employ techniques similar to those for expression of immunoglobulin heavy chain libraries described in U.S. Pat. No. 5,824,520. Previously employed Fc libraries are discussed in PCT Publn. WO 2008/137475, which is specifically incorporated herein by reference. In some embodiments, yeast surface display libraries are used (e.g., Choi et al. 2015; Wozniak-Knopp et al., 2010).

### II. ANTIBODY-BINDING POLYPEPTIDES

A variety of antibody-binding domains (e.g., FcR polypeptides) are known in the art and may be used in the methods and compositions of the invention. For example, in some aspects, an FcR may have specificity for a particular type or subtype of Ig, such as IgA, IgM, IgE, or IgG (e.g., IgG1, IgG2a, IgG2b, IgG3, or IgG4). Thus, in some embodiments the antibody-binding domain may be defined as an IgG binding domain. The FcR polypeptide may comprise a eukaryotic, prokaryotic, or synthetic FcR domain. For instance, an antibody Fc-binding domain may be defined as a mammalian, bacterial, or synthetic binding domain. Some Fc-binding domains for use in the invention include but are not limited to a binding domain from one of the polypeptides of Table 3. For example, an Fc-binding polypeptide may be encoded by an FCGR2A, FCGR2B, FCGR2C, FCGR3A, FCGR3B, FCGR1A, Fcgr1, FCGR2, FCGR2, Fcgr2, Fcgr2, FCGR3, FCGR3, Fcgr3, FCGR3, Fcgr3, FCGRT, mrp4, spa, or spg gene. An FcR polypeptide may be an Fc binding region from human FcγRIA, FcγRIIA, FcγRIIB, FcγRIIc, FcγRIIIA, FcγRIIIb, FcαRI, or C1q. A variety of Fc receptors to which Fc domains bind are well known in the art and some examples of receptors are listed below in Table 3.

TABLE 3
Selected FcR Polypeptides

| | | | | Length | |
|---|---|---|---|---|---|
| Protein name | Gene name | Description | Organisms | (aa) | Reference |
| Fc-gamma | FCGR2A | Low affinity immunoglobulin | Homo sapiens | 317 | (Stuart et al., |
| RII-a (CD32) | | gamma Fc region receptor II-a | (Human) | | 1987) |
| | | precursor | | | |
| Fc-gamma | FCGR2A | Low affinity immunoglobulin | Pan troglodytes | 316 | |
| RII-a | | gamma Fc region receptor II-a | (Chimpanzee) | | |
| | | precursor | | | |
| Fc-gamma | FCGR2B | Low affinity immunoglobulin | Homo sapiens | 310 | (Stuart et al., |
| RII-b | | gamma Fc region receptor II-b | (Human) | | 1989) |
| | | precursor | | | |
| Fc-gamma | FCGR2C | Low affinity immunoglobulin | Homo sapiens | 323 | (Stuart et al., |
| RII-c | | gamma Fc region receptor II-c | (Human) | | 1989) |
| | | precursor | | | |
| Fc-gamma | FCGR3A | Low affinity immunoglobulin | Homo sapiens | 254 | (Ravetch and |
| RIIIa | | gamma Fc region receptor III-A | (Human) | | Perussia, |
| | | precursor | | | 1989) |
| Fc-gamma | FCGR3B | Low affinity immunoglobulin | Homo sapiens | 233 | (Ravetch and |
| RIIIb | | gamma Fc region receptor III-B | (Human) | | Perussia, |
| | | precursor | | | 1989) |
| Fc-gamma RI | FCGR1A | High affinity immunoglobulin | Homo sapiens | 374 | (Allen and |
| (CD64) | | gamma Fc receptor I precursor | (Human) | | Seed, 1988) |
| Fc-gamma RI | Fcgr1 | High affinity immunoglobulin | Mus musculus | 404 | (Sears et al., |
| | | gamma Fc receptor I precursor | (Mouse) | | 1990) |
| Fc-gamma | FCGR2 | Low affinity immunoglobulin | Bos taurus | 296 | (Zhang et al., |
| RII | | gamma Fc region receptor II | (Bovine) | | 1994) |
| | | precursor | | | |
| Fc-gamma | FCGR2 | Low affinity immunoglobulin | Cavia porcellus | 341 | (Tominaga et |
| RII | | gamma Fc region receptor II | (Guinea pig) | | al., 1990) |
| | | precursor | | | |
| Fc-gamma | Fcgr2 | Low affinity immunoglobulin | Mus musculus | 330 | (Ravetch et |
| RII | | gamma Fc region receptor II | (Mouse) | | al., 1986) |
| | | precursor | | | |
| Fc- gamma | Fcgr2 | Low affinity immunoglobulin | Rattus norvegicus | 285 | (Bocek and |
| RII | | gamma Fc region receptor II | (Rat) | | Pecht, 1993) |
| | | precursor | | | |
| Fc-gamma | FCGR3 | Low affinity immunoglobulin | Bos taurus | 250 | (Collins et |
| RIII | | gamma Fc region receptor III | (Bovine) | | al., 1997) |
| | | precursor | | | |
| Fc-gamma | FCGR3 | Low affinity immunoglobulin | Macaca | 254 | |
| RIII | | gamma Fc region receptor III | fascicularis (Crab | | |
| | | precursor | eating macaque) | | |
| | | | (Cynomolgus | | |
| | | | monkey) | | |
| Fc-gamma | Fcgr3 | Low affinity immunoglobulin | Mus musculus | 261 | (Ravetch et |
| RIII | | gamma Fc region receptor III | (Mouse) | | al., 1986) |
| | | precursor | | | |
| Fc-gamma | FCGR3 | Low affinity immunoglobulin | Sus scrofa (Pig) | 257 | (Halloran et |
| RIII | | gamma Fc region receptor III | | | al., 1994) |
| | | precursor | | | |
| Fc-gamma | Fcgr3 | Low affinity immunoglobulin | Rattus norvegicus | 267 | (Zeger et al., |
| RIII | | gamma Fc region receptor III | (Rat) | | 1990) |
| | | precursor | | | |
| FcRn | FCGRT | IgG receptor transporter FcRn | Homo sapiens | 365 | |
| | | large subunit p51 precursor | (Human) | | |
| FcRn | FCGRT | IgG receptor transporter FcRn | Macaca | 365 | |
| | | large subunit p51 precursor | fascicularis (Crab | | |
| | | | eating macaque) | | |
| | | | (Cynomolgus | | |
| | | | monkey) | | |
| FcRn | Fcgrt | IgG receptor transporter FcRn | Mus musculus | 365 | (Ahouse et |
| | | large subunit p51 precursor | (Mouse) | | al., 1993) |
| FcRn | Fcgrt | IgG receptor transporter FcRn | Rattus norvegicus | 366 | (Simister and |
| | | large subunit p51 precursor | (Rat) | | Mostov, |
| | | | | | 1989) |
| MRP protein | mrp4 | Fibrinogen- and Ig-binding | Streptococcus | 388 | (Stenberg et |
| | | protein precursor | pyogenes | | al., 1992) |
| Protein B | | cAMP factor | Streptococcus | 226 | (Ruhlmann |
| | | | agalactiae | | et al., 1988) |
| protein A | spa | Immunoglobulin G-binding | Staphylococcus | 516 | (Uhlen et al., |
| | | protein A precursor | aureus (strain | | 1984) |
| | | | NCTC 8325) | | |
| protein A | spa | Immunoglobulin G-binding | Staphylococcus | 508 | (Shuttleworth |
| | | protein A precursor | aureus | | et al., |
| | | | | | 1987) |
| protein A | spa | Immunoglobulin G-binding | Staphylococcus | 450 | (Kuroda et |
| | | protein A precursor | aureus (strain | | al., 2001) |
| | | | Mu50/ATCC | | |
| | | | 700699) | | |
| protein A | spa | Immunoglobulin G-binding | Staphylococcus | 450 | (Kuroda et |
| | | protein A precursor | aureus (strain | | al., 2001) |
| | | | N315) | | |
| protein G | spg | Immunoglobulin G-binding | Streptococcus sp. | 448 | (Fahnestock |
| | | protein G precursor | group G | | et al., 1986) |
| protein G | spg | Immunoglobulin G-binding | Streptococcus sp. | 593 | (Olsson et |
| | | protein G precursor | group G | | al., 1987) |
| protein H | | Immunoglobulin G-binding | Streptococcus | 376 | (Gomi et al., |
| | | protein H precursor | pyogenes serotype | | 1990) |
| | | | M1 | | |
| Protein sbi | sbi | Immunoglobulin G-binding | Staphylococcus | 436 | (Zhang et al., |
| | | protein sbi precursor | aureus (strain | | 1998) |
| | | | NCTC 8325-4) | | |
| Allergen Asp | | Allergen Asp fl 1 causes an | Aspergillus flavus | 32 | |
| fl 1 | | allergic reaction in human. Binds | | | |
| | | to IgE and IgG | | | |
| Allergen Asp | | Allergen Asp fl 2 causes an | Aspergillus flavus | 20 | |
| fl 2 | | allergic reaction in human. Binds | | | |
| | | to IgE and IgG | | | |
| Allergen Asp | | Allergen Asp fl 3 causes an | Aspergillus flavus | 32 | |
| fl 3 | | allergic reaction in human. Binds | | | |
| | | to IgE and IgG | | | |
| Fc-epsilon RI | | IgE receptor displayed on Mast | Homo sapiens | | |
| | | cells, Eosinophils and Basophils | (Human) | | |
| Fc-alpha RI | | IgA (IgA1, IgA2) receptor | Homo sapiens | | |
| (CD86) | | displayed on Macrophages | (Human) | | |
| C1q | C1QA | C1q is multimeric complex that | Homo sapiens | | |
| | NP_057075.1, | binds to antibody Fc composed of | (Human) | | |
| | C1QB | 6 A chains, 6 B chains and 6 C | | | |
| | NP_000482.3, | chains | | | |
| | C1QC | | | | |
| | NP_758957.1 | | | | |

### III. METHODS FOR SCREENING ANTIBODY FC DOMAINS

In certain aspects there are methods for identifying antibody Fc domains with a specific affinity for a target ligand (e.g., an antibody-binding polypeptide, such as an Fc receptor). Such methods are described herein, as well as in PCT Publn. WO 2008/137475, which is hereby specifically incorporated by reference in its entirety. In some embodiments, methods of screening using eukaryotic cells (e.g., yeast surface display libraries) can be used.

The polypeptides screened may comprise a large library of diverse candidate Fc domains, or, alternatively, may comprise particular classes of Fc domains (e.g., engineered point mutations or amino acid insertions) selected with an eye towards structural attributes that are believed to make them more likely to bind the target ligand. In one embodiment, the candidate polypeptide may be an intact antibody, or a fragment or portion thereof comprising an Fc domain.

To identify a candidate Fc domain capable of binding a target ligand, one may carry out the steps of: providing a population of Gram-negative bacterial cells that each expresses a distinct antibody Fc domain; admixing the bacteria and at least a first labeled or immobilized target ligand (FcR polypeptide) capable of contacting the antibody Fc domain; and identifying at least a first bacterium expressing a molecule capable of binding the target ligand.

In some aspects of the aforementioned method, the binding between antibody Fc domain and a labeled FcR polypeptide will prevent diffusion out of a bacterial cell. In this way, molecules of the labeled ligand can be retained in the periplasm of the bacterium comprising a permeabilized outer membrane. Alternatively, the periplasm can be removed, whereby the Fc domain will cause retention of the bound candidate molecule since Fc domains are shown to associate with the inner membrane. The labeling may then be used to isolate the cell expressing a binding polypeptide capable of binding the FcR polypeptide, and the gene encoding the Fc domain polypeptide may be isolated. The molecule capable of binding the target ligand may then be produced in large quantities using in vivo or ex vivo expression methods, and then used for any desired application, for example, for diagnostic or therapeutic applications. Furthermore, it will be understood that isolated antibody Fc domains identified may be used to construct an antibody fragment or full-length antibody comprising an antigen binding domain.

In further embodiments, methods of screening may comprise at least two rounds of selection wherein the sub-population of bacterial cells obtained in the first round of selection is subjected to at least a second round of selection based on the binding of the candidate antibody Fc domain to an FcR. The sub-population of bacterial cells obtained in the first round of selection may be grown under permissive conditions prior to a second selection (to expand the total number of cells). The methods may for example comprise 2, 3, 4, 5, 6, 7, 8, 9, 10 or more rounds of selection. In some aspects, a sub-population of bacterial cells obtained from each round of selection will be grown under permissive conditions before a subsequent round of selection. Cells isolated following one or more such rounds of selection may be subjected to additional rounds of mutagenesis. In some cases, selection will be performed after removing FcR polypeptide that is not bound to the antibody. The stringency of selection may be modified by adjusting the pH, salt concentration, or temperature of a solution comprising bacteria that display antibodies. For example, a bacterial cell may be grown at a sub-physiological temperature, such as at about 25° C.

Methods of producing bacterial cells are provided, and the bacterial cell may comprise a nucleic acid sequence encoding a mutant Fc domain provided herein. A bacterial cell produced by methods provided herein may be used to clone a nucleic acid sequence encoding the Fc domain having a specific affinity for an FcR polypeptide. Methods for isolating and amplifying such a nucleic acid from a cell, for example by PCR are well known in the art and further described below. Nucleic acid sequences produced by the foregoing methods comprise aspects of the present disclosure. The nucleic acid can be expressed in a cell to produce an Fc domain provided herein or a polypeptide comprising an Fc domain provided herein. In some embodiments. an antibody Fc domain provided herein is comprised in a polypeptide with one or more antibody variable region(s) (e.g., single domain antibody, scFv, etc.) that have an affinity for a particular target ligand.

B. Periplasmic Expression of Antibody Fc Domains

In some embodiments, a polypeptide comprising an antibody Fc domain may be expressed in the periplasmic space of Gram-negative bacteria. Furthermore, in some aspects an antibody Fc domain may be anchored to the periplasmic face of the inner membrane. Methods and compositions for the anchoring of polypeptides to the inner membrane of Gram-negative bacteria have previously been described (U.S. Pat. Nos. 7,094,571, 7,419,783, 7,611,866 and U.S. Patent Publn. No. 2003/0219870; Harvey et al., 2004; Harvey et al., 2006). For example, an Fc domain may be directly fused to a membrane spanning or membrane bound polypeptide or may interact (e.g., via protein-protein interactions) with a membrane spanning or membrane bound polypeptide. This technique may be termed “Anchored Periplasmic Expression” or “APEx.” In some cases, a Gram-negative bacterial cell may be defined as an E. coli cell. Furthermore, in some aspects a Gram-negative bacterial cell may be defined as a genetically engineered bacterial cell, such as a Jude-1 strain of E. coli.

A fusion protein may comprise an N-terminal or C-terminal fusion with an Fc domain and in some cases may comprise additional linker amino acids between the membrane anchoring polypeptide and the Fc domain. In certain specific cases, a membrane anchoring polypeptide may be the first six amino acids encoded by the E. coli NlpA gene, one or more transmembrane α-helices from an E. coli inner membrane protein, a gene III protein of filamentous phage or a fragment thereof, or an inner membrane lipoprotein or fragment thereof. Thus, as an example, a membrane anchoring polypeptide may be an inner membrane lipoprotein or fragment thereof such as from AraH, MglC, MalF, MalG, MalC, MalD, RbsC, RbsC, ArtM, ArtQ, GlnP, ProW, HisM, HisQ, LivH, LivM, LivA, LivE, DppB, DppC, OppB, AmiC, AmiD, BtuC, ThuD, FecC, FecD, FecR, FepD, NikB, NikC, CysT, CysW, UgpA, UgpE, PstA, PstC, PotB, PotC, PotH, Pod, ModB, NosY, PhnM, LacY, SecY, TolC, DsbB, DsbD, TouB, TatC, CheY, TraB, ExbD, ExbB, or Aas.

In still further cases, a population of Gram-negative bacteria according to the invention may be defined as comprising at least about 1×10³, 1×10⁴, 1×10⁵, 1×10⁶, 1×10⁷, 1×10⁸, 1×10⁹ or more distinct antibodies Fc domains. In some specific cases, a population of Gram-negative bacterial cells may be produced by a method comprising the steps of: (a) preparing a plurality of nucleic acid sequences encoding a plurality of distinct antibody Fc domains; and (b) transforming a population of Gram-negative bacteria with said nucleic acids wherein the Gram-negative bacteria comprise a plurality of antibody Fc domains expressed in the periplasm.

C. Permeabilization of the Outer Membrane

Methods for disrupting, permeabilizing, or removing the outer membrane of bacteria are well known in the art, for example, see U.S. Pat. No. 7,094,571. For instance, prior to contacting the bacterial cells with an FcR polypeptide, the outer membrane of the bacterial cell may be treated with hyperosmotic conditions, physical stress, lysozyme, EDTA, a digestive enzyme, a chemical that disrupts the outer membrane, by infecting the bacterium with a phage, or a combination of the foregoing methods. Thus, in some cases, the outer membrane may be disrupted by lysozyme and EDTA treatment. Furthermore, in certain embodiments, the bacterial outer membrane may be removed entirely.

Methods may be employed for increasing the permeability of the outer membrane to one or more labeled ligands. This can allow screening access of labeled ligands otherwise unable to cross the outer membrane. However, certain classes of molecules, for example, hydrophobic antibiotics larger than the 650 Da exclusion limit, can diffuse through the bacterial outer membrane itself, independent of membrane porins (Farmer et al., 1999). The process may permeabilize the membrane (Jouenne and Junter, 1990). Also, certain long chain phosphate polymers (100 Pi) appear to bypass the normal molecular sieving activity of the outer membrane altogether (Rao and Torriani, 1988).

While conditions have been identified that lead to the permeation of ligands into the periplasm without loss of viability or release of the expressed proteins from the cells, the invention may be carried out without maintaining the outer membrane. For Fc domains expressed or anchored in the periplasmic space, the need to maintain the outer membrane (as a barrier to prevent the leakage of the binding protein from the cell) to detect bound labeled ligand is removed. As a result, cells expressing binding proteins anchored to the outer (periplasmic) face of the cytoplasmic membrane can be labeled simply by incubating with a solution of labeled ligand with cells that either have a partially permeabilized membrane or a nearly completely removed outer membrane.

Treatments, such as hyperosmotic shock, can improve labeling significantly. It is known that many agents, including calcium ions (Bukau et al., 1985) and even Tris buffer (Irvin et al., 1981), alter the permeability of the outer-membrane. Further, phage infection stimulates the labeling process. Both the filamentous phage inner membrane protein pIII and the large multimeric outer membrane protein pIV can alter membrane permeability (Boeke et al., 1982) with mutants in pIV known to improve access to maltodextrins normally excluded (Marciano et al., 1999). Combinations of strain, salt, and phage can be used to achieve a high degree of permeability (Daugherty et al., 1999). Cells comprising anchored or periplasm-associated polypeptides bound to labeled ligands can then be easily isolated from cells that express binding proteins without affinity for the labeled ligand using flow cytometry or other related techniques. However, in some cases, it will be desired to use less disruptive techniques in order to maintain the viability of cells. EDTA and lysozyme treatments may also be useful in this regard.

D. Labeled Target Ligands

As indicated above, it will typically be desired to provide an FcR polypeptide that has been labeled with one or more detectable agent(s). This can be carried out, for example, by linking the ligand to at least one detectable agent to form a conjugate. For example, it is conventional to link or covalently bind or complex at least one detectable molecule or moiety. A “label” or “detectable label” is a compound and/or element that can be detected due to specific functional properties, and/or chemical characteristics, the use of which allows the ligand to which it is attached to be detected, and/or further quantified if desired. Examples of labels that could be used include, but are not limited to, enzymes, radiolabels, haptens, fluorescent labels, phosphorescent molecules, chemiluminescent molecules, chromophores, luminescent molecules, photoaffinity molecules, colored particles, or ligands, such as biotin. In some embodiments, a streptavidin-biotinylated FcgR tetramer can be used for screening, and FcgR with Avi-tag can be used for biotinylation.

In one embodiment of the invention, a visually-detectable marker is used such that automated screening of cells for the label can be carried out. Examples of agents that may be detected by visualization with an appropriate instrument are known in the art, as are methods for their attachment to a desired ligand (see, e.g., U.S. Pat. Nos. 5,021,236; 4,938,948; and 4,472,509, each incorporated herein by reference). Such agents can include paramagnetic ions; radioactive isotopes; fluorochromes; NMR-detectable substances; and substances for X-ray imaging. In particular, fluorescent labels are beneficial in that they allow use of flow cytometry for isolation of cells expressing a desired binding protein or antibody.

Another type of FcR conjugate is where the ligand is linked to a secondary binding molecule and/or to an enzyme (an enzyme tag) that will generate a colored product upon contact with a chromogenic substrate. Examples of such enzymes include urease, alkaline phosphatase, (horseradish) hydrogen peroxidase, or glucose oxidase. In such instances, it will be desired that cells selected remain viable. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149; and 4,366,241, each incorporated herein by reference.

Molecules containing azido groups may be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide-binding proteins in crude cell extracts (Owens and Haley, 1987; Atherton et al., 1985). The 2- and 8-azido nucleotides have also been used to map nucleotide-binding domains of purified proteins (Khatoon et al., 1989; King et al., 1989; Dholakia et al., 1989) and may be used as ligand binding agents.

Labeling can be carried out by any of the techniques well known to those of skill in the art. For instance, FcR polypeptides can be labeled by contacting the ligand with the desired label and a chemical oxidizing agent, such as sodium hypochlorite, or an enzymatic oxidizing agent, such as lactoperoxidase. Similarly, a ligand exchange process could be used. Alternatively, direct labeling techniques may be used, e.g., by incubating the label, a reducing agent such as SNCl₂, a buffer solution such as sodium-potassium phthalate solution, and the ligand. Intermediary functional groups on the ligand could also be used, for example, to bind labels to a ligand in the presence of diethylenetriaminepentaacetic acid (DTPA) or ethylene diaminetetracetic acid (EDTA).

Other methods are also known in the art for the attachment or conjugation of a ligand to its conjugate moiety. Some attachment methods involve the use of an organic chelating agent, such as diethylenetriaminepentaacetic acid anhydride (DTPA); ethylenetriaminetetraacetic acid or ethylenediaminetetraacetic acid; N-chloro-p-toluenesulfonamide; and/or tetrachloro-3α-6α-diphenylglycouril-3 attached to the ligand (U.S. Pat. Nos. 4,472,509 and 4,938,948, each incorporated herein by reference). FcR polypeptides also may be reacted with an enzyme in the presence of a coupling agent such as glutaraldehyde or periodate. Conjugates with fluorescein markers can be prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate. In still further aspects an FcR polypeptide may be fused to a reporter protein, such as an enzyme as described supra or a fluorescence protein.

E. Isolation of Cells Bound to Labeled Target Ligand

1. Column- or Bead-Based Immobilization

The skilled artisan will understand that methods for selecting cells based upon their interaction (binding) with an FcR are well-known in the art. For example, an FcR may be immobilized on a column or bead (e.g., a magnetic bead) and the cell (e.g., bacterial cell, or eukaryotic cell such as a yeast) binding to the FcR separated by repeated washing of the bead (e.g., magnetic separation) or column. Furthermore, a target ligand may be labeled (e.g., with a fluorophore, a radioisotope, or an enzyme). Thus, the cells may, in some cases, be selected by detecting a label on a bound FcR. Furthermore, in some aspects, the cells may be selected based on binding or lack of binding to two or more FcR polypeptides. For instance, bacteria may be selected that display antibodies that bind to two FcR polypeptides, wherein each FcR is used to select the bacteria sequentially. Conversely, in certain aspects, bacteria may be selected that display antibody Fc domains that bind to one FcR (such as an FcR comprising a first label) but not to a second FcR (e.g., comprising a second label). The foregoing method may be used, for example, to identify antibody Fc domains that bind to a specific FcR but not a second specific FcR.

2. Flow Cytometry

In one embodiment of the invention, fluorescence activated cell sorting (FACS) screening or other automated flow cytometric techniques may be used for the efficient isolation of a bacterial cell comprising a labeled ligand bound to an Fc domain. Instruments for carrying out flow cytometry are known to those of skill in the art and are commercially available to the public. Examples of such instruments include FACS Star Plus, FACScan and FACSort instruments from Becton Dickinson (Foster City, CA), Epics C from Coulter Epics Division (Hialeah, FL), and MOFLO™ from Cytomation (Colorado Springs, CO).

Flow cytometric techniques in general involve the separation of cells or other particles in a liquid sample. Typically, the purpose of flow cytometry is to analyze the separated particles for one or more characteristics thereof, for example, presence of a labeled ligand or other molecule. The basic steps of flow cytometry involve the direction of a fluid sample through an apparatus such that a liquid stream passes through a sensing region. The particles should pass one at a time by the sensor and are categorized based on size, refraction, light scattering, opacity, roughness, shape, fluorescence, etc.

Not only is cell analysis performed by flow cytometry, but so too is sorting of cells. In U.S. Pat. No. 3,826,364, an apparatus is disclosed which physically separates particles, such as functionally different cell types. In this machine, a laser provides illumination that is focused on the stream of particles by a suitable lens or lens system so that there is highly localized scatter from the particles therein. In addition, high intensity source illumination is directed onto the stream of particles for the excitation of fluorescent particles in the stream. Certain particles in the stream may be selectively charged and then separated by deflecting them into designated receptacles. A classic form of this separation is via fluorescent-tagged antibodies, which are used to mark one or more cell types for separation.

Other examples of methods for flow cytometry include, but are not limited to, those described in U.S. Pat. Nos. 4,284,412; 4,989,977; 4,498,766; 4,857,451; 4,774,189; 4,767,206; 4,714,682; 5,160,974; 5,478,722; and 4,661,913, each of which are specifically incorporated herein by reference.

A useful aspect of flow cytometry is that multiple rounds of screening can be carried out sequentially. Cells may be isolated from an initial round of sorting and immediately reintroduced into the flow cytometer and screened again to improve the stringency of the screen. Another advantage known to those of skill in the art is that nonviable cells can be recovered using flow cytometry. Since flow cytometry is essentially a particle sorting technology, the ability of a cell to grow or propagate is not necessary. Techniques for the recovery of nucleic acids from such non-viable cells are well known in the art and include, for example, use of template-dependent amplification techniques including PCR.

F. Cloning of Fc Domain Coding Sequences

After a bacterial cell is identified that produces molecules of the desired specificity, affinity, and/or activity, the corresponding coding sequence may be cloned. In this manner, DNA encoding the molecule can be isolated and sequenced using conventional procedures (e.g., by using oligonucleotide probes that are capable of binding specifically to genes encoding the antibody or binding protein). It will be understood by those of skill in the art that nucleic acids may be cloned from viable or inviable cells. In the case of inviable cells, for example, it may be desired to use amplification of the cloned DNA, for example, using PCR. This may also be carried out using viable cells either with or without further growth of cells.

Once isolated, the antibody Fc domain DNA may be placed into expression vectors, which can then be transfected into host cells, such as bacteria. The DNA also may be modified, for example, by the addition of sequence for human heavy and light chain variable domains, or by covalently joining to the immunoglobulin coding sequence all or part of the coding sequence for a non-immunoglobulin polypeptide. In that manner, “chimeric” or “hybrid” binding proteins are prepared to have the desired binding specificity. For instance, an identified antibody Fc domain may be fused to a therapeutic polypeptide or a toxin and used to target cells (in vitro or in vivo) that express a particular FcR.

Chimeric or hybrid Fc domains also may be prepared in vitro using known methods in synthetic protein chemistry, including those involving crosslinking agents. For example, targeted-toxins may be constructed using a disulfide exchange reaction or by forming a thioether bond. Examples of suitable reagents for this purpose include iminothiolate and methyl-4-mercaptobutyrimidate.

### IV. NUCLEIC ACID-BASED EXPRESSION SYSTEMS

Nucleic acid-based expression systems may find use, in certain embodiments of the invention, for the expression of recombinant proteins. For example, one embodiment of the invention involves transformation of Gram-negative bacteria with the coding sequences for an antibody Fc domain, or preferably a plurality of distinct Fc domains.

A. Methods of Nucleic Acid Delivery

Certain aspects of the invention may comprise delivery of nucleic acids to target cells (e.g., Gram-negative bacteria). For example, bacterial host cells may be transformed with nucleic acids encoding candidate Fc domains potentially capable binding an FcR. In particular embodiments of the invention, it may be desired to target the expression to the periplasm of the bacteria. Transformation of eukaryotic host cells may similarly find use in the expression of various candidate molecules identified as capable of binding a target ligand.

Suitable methods for nucleic acid delivery for transformation of a cell are believed to include virtually any method by which a nucleic acid (e.g., DNA) can be introduced into a cell, or even an organelle thereof. Such methods include, but are not limited to, direct delivery of DNA, such as by injection (U.S. Pat. Nos. 5,994,624; 5,981,274; 5,945,100; 5,780,448; 5,736,524; 5,702,932; 5,656,610; 5,589,466; and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Publn. Nos. WO 94/09699 and 95/06128; U.S. Pat. Nos. 5,610,042; 5,322,783; 5,563,055; 5,550,318; 5,538,877; and 5,538,880, and each incorporated herein by reference); or by agitation with silicon carbide fibers (Kaeppler et al., 1990; U.S. Pat. Nos. 5,302,523 and 5,464,765, each incorporated herein by reference); by desiccation/inhibition-mediated DNA uptake (Potrykus et al., 1985). Through the application of techniques such as these, cells may be stably or transiently transformed.

B. Vectors

Vectors may find use with the current invention, for example, in the transformation of a cell with a nucleic acid sequence encoding a candidate Fc domain. In one embodiment of the invention, an entire heterogeneous “library” of nucleic acid sequences encoding polypeptides may be introduced into a population of cells, thereby allowing screening of the entire library. The term “vector” is used to refer to a carrier nucleic acid molecule into which a nucleic acid sequence can be inserted for introduction into a cell where it can be replicated. A nucleic acid sequence can be “exogenous” or “heterologous,” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (e.g., bacteriophage). One of skill in the art may construct a vector through standard recombinant techniques, which are described in Maniatis et al., 1988 and Ausubel et al., 1994, both of which are incorporated herein by reference.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well.

1. Promoters and Enhancers

A “promoter” is a control sequence that is a region of a nucleic acid sequence at which initiation and rate of transcription are controlled. It may contain genetic elements to which regulatory proteins and molecules may bind, such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence. Those of skill in the art of molecular biology generally are familiar with the use of promoters, enhancers, and cell type combinations for protein expression, for example, see Sambrook et al. (1989), incorporated herein by reference.

2. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

3. Multiple Cloning Sites

Vectors can include a multiple cloning site (MCS), which is a nucleic acid region that contains multiple restriction enzyme sites, any of which can be used in conjunction with standard recombinant technology to digest the vector (see Carbonelli et al., 1999, Levenson et al., 1998, and Cocea, 1997, incorporated herein by reference). “Restriction enzyme digestion” refers to catalytic cleavage of a nucleic acid molecule with an enzyme that functions only at specific locations in a nucleic acid molecule. Many of these restriction enzymes are commercially available. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Termination Signals

The vectors or constructs prepared in accordance with the present disclosure will generally comprise at least one termination signal. A “termination signal” or “terminator” is comprised of the DNA sequences involved in specific termination of an RNA transcript by an RNA polymerase. Thus, in certain embodiments, a termination signal that ends the production of an RNA transcript is contemplated. A terminator may be necessary in vivo to achieve desirable message levels. Terminators contemplated for use in the invention include any known terminator of transcription known to one of ordinary skill in the art, including, but not limited to, rho dependent or rho independent terminators. In certain embodiments, the termination signal may be a lack of transcribable or translatable sequence, such as due to a sequence truncation.

5. Origins of Replication

In order to propagate a vector in a host cell, it may contain one or more origins of replication sites (often termed “ori”), which is a specific nucleic acid sequence at which replication is initiated.

6. Selectable and Screenable Markers

In certain embodiments of the invention, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually, the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers, such as GFP, whose basis is colorimetric analysis, are also contemplated. Alternatively, screenable enzymes such as chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

C. Host Cells

In the context of expressing a heterologous nucleic acid sequence, “host cell” refers to a prokaryotic cell or a eukaryotic cell (e.g., a yeast cell, an insect cell, or a mammalian cell), and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

In particular embodiments of the invention, a host cell is a Gram-negative bacterial cell. These bacteria are suited for use with the invention in that they possess a periplasmic space between the inner and outer membrane and, particularly, the aforementioned inner membrane between the periplasm and cytoplasm, which is also known as the cytoplasmic membrane. As such, any other cell with such a periplasmic space could be used in accordance with the invention. Examples of Gram-negative bacteria that may find use with the invention may include, but are not limited to, E. coli, Pseudomonas aeruginosa, Vibrio cholera, Salmonella typhimurium, Shigella flexneri, Haemophilus influenza, Bordotella pertussi, Erwinia amylovora, Rhizobium sp.

An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (Stratagene®, La Jolla). Alternatively, bacterial cells such as E. coli LE392 could be used as host cells for bacteriophage.

Examples of mammalian host cells include Chinese hamster ovary cells (CHO-K1; ATCC CCL61), rat pituitary cells (GH1; ATCC CCL82), HeLa S3 cells (ATCC CCL2.2), rat hepatoma cells (H-4-II-E; ATCCCRL 1548), SV40-transformed monkey kidney cells (COS-1; ATCC CRL 1650), murine embryonic cells (NIH-3T3; ATCC CRL 1658), and human embryonic kidney cells (e.g., EXPI293 cells). The foregoing being illustrative but not limitative of the many possible host organisms known in the art.

Mammalian host cells expressing the polypeptide are cultured under conditions typically employed to culture the parental cell line. Generally, cells are cultured in a standard medium containing physiological salts and nutrients, such as standard RPMI, MEM, IMEM, or DMEM, typically supplemented with 5%-10% serum, such as fetal bovine serum. Culture conditions are also standard, e.g., cultures are incubated at 37° C. in stationary or roller cultures until desired levels of the proteins are achieved.

Many host cells from various cell types and organisms are available and would be known to one of skill in the art. Similarly, a viral vector may be used in conjunction with a prokaryotic host cell, particularly one that is permissive for replication or expression of the vector. Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

D. Expression Systems

Numerous expression systems exist that comprise at least a part or all of the compositions discussed above. Such systems could be used, for example, for the production of a polypeptide product identified in accordance with the invention as capable of binding a particular ligand. Prokaryote-based systems can be employed for use with the present disclosure to produce nucleic acid sequences, or their cognate polypeptides, proteins, and peptides. Many such systems are commercially and widely available. Other examples of expression systems comprise of vectors containing a strong prokaryotic promoter such as T7, Tac, Trc, BAD, lambda pL, Tetracycline or Lac promoters, the pET Expression System, and an E. coli expression system.

In certain aspects of the invention, nucleic acid sequences encoding a polypeptide are disclosed. Depending on which expression system is used, nucleic acid sequences can be selected based on conventional methods. For example, if the polypeptide is derived from a human polypeptide and contains multiple codons that are rarely utilized in E. coli, then that may interfere with expression in E. coli. Therefore, the respective genes or variants thereof may be codon optimized for E. coli expression. Various vectors may be also used to express the protein of interest. Exemplary vectors include, but are not limited, plasmid vectors, viral vectors, transposon, or liposome-based vectors.

### V. PROTEIN PURIFICATION

Protein purification techniques are well known to those of skill in the art. These techniques involve, at one level, the homogenization and crude fractionation of the cells, tissue, or organ into polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure peptide are ion-exchange chromatography, size-exclusion chromatography, reverse phase chromatography, hydroxyapatite chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC). As is generally known in the art, it is believed that the order of conducting the various purification steps may be changed, or that certain steps may be omitted, and still result in a suitable method for the preparation of a substantially purified protein or peptide.

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “fold purification number.” The actual units used to represent the amount of activity will, of course, be dependent upon the particular assay technique chosen to follow the purification, and whether or not the expressed protein or peptide exhibits a detectable activity.

There is no general requirement that the protein or peptide will always be provided in its most purified state. Indeed, it is contemplated that less substantially purified products may have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding. It should be possible to elute the substance without destroying the sample or the ligand.

Size-exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase. The underlying principle of SEC is that particles of different sizes will elute (filter) through a stationary phase at different rates. This results in the separation of a solution of particles based on size. Provided that all the particles are loaded simultaneously or near simultaneously, particles of the same size should elute together.

High-performance liquid chromatography (or high-pressure liquid chromatography, HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used.

### VI. PHARMACEUTICAL COMPOSITIONS

Where clinical application of a pharmaceutical composition containing a polypeptide or antibody is undertaken, it will generally be beneficial to prepare a pharmaceutical or therapeutic composition appropriate for the intended application. Generally, pharmaceutical compositions may comprise an effective amount of one or more polypeptide or additional agents dissolved or dispersed in a pharmaceutically acceptable carrier. In certain embodiments, pharmaceutical compositions may comprise, for example, at least about 0.1% of a polypeptide or antibody. In other embodiments, a polypeptide or antibody may comprise between about 2% to about 75% of the weight of the unit, or between about 25% to about 60%, for example, and any range derivable therein. The amount of active compound(s) in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by FDA Office of Biological Standards.

Further in accordance with certain aspects of the present invention, the composition suitable for administration may be provided in a pharmaceutically acceptable carrier with or without an inert diluent. The carrier should be assimilable and includes liquid, semi-solid, i.e., pastes, or solid carriers. Examples of carriers or diluents include fats, oils, water, saline solutions, lipids, liposomes, resins, binders, fillers, and the like, or combinations thereof. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal), isotonic agents (e.g., sugars, sodium chloride), absorption delaying agents (e.g., aluminum monostearate, gelatin), salts, drugs, drug stabilizers (e.g., buffers, amino acids, such as glycine and lysine, carbohydrates, such as dextrose, mannose, galactose, fructose, lactose, sucrose, maltose, sorbitol, mannitol, etc), gels, binders, excipients, disintegration agents, lubricants, sweetening agents, flavoring agents, dyes, fluid and nutrient replenishers, such like materials and combinations thereof, as would be known to one of ordinary skill in the art. Except insofar as any conventional media, agent, diluent, or carrier is detrimental to the recipient or to the therapeutic effectiveness of the composition contained therein, its use in administrable composition for use in practicing the methods is appropriate. The pH and exact concentration of the various components in a pharmaceutical composition are adjusted according to well-known parameters. The composition may be combined with the carrier in any convenient and practical manner, i.e., by solution, suspension, emulsification, admixture, encapsulation, absorption, grinding, and the like. Such procedures are routine for those skilled in the art.

Certain embodiments of the present disclosure may comprise different types of carriers depending on whether it is to be administered in solid, liquid, or aerosol form, and whether it needs to be sterile for the route of administration, such as injection. The compositions can be formulated for administration intravenously, intradermally, transdermally, intrathecally, intraarterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference). Typically, such compositions can be prepared as either liquid solutions or suspensions; solid forms suitable for use to prepare solutions or suspensions upon the addition of a liquid prior to injection can also be prepared; and, the preparations can also be emulsified.

The polypeptides may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, histidine, or procaine.

In further embodiments, the present disclosure may concern the use of a pharmaceutical lipid vehicle composition that includes polypeptides, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods.

One of ordinary skill in the art would be familiar with the range of techniques that can be employed for dispersing a composition in a lipid vehicle. For example, the polypeptide or a fusion protein thereof may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above. The practitioner responsible for administration will, in any event, determine the concentration of active ingredient(s) in a composition and appropriate dose(s) for the individual subject.

It is not intended that the present invention be limited by the particular nature of the therapeutic preparation. For example, such compositions can be provided in formulations together with physiologically tolerable liquid, gel, or solid carriers, diluents, and excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects. The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

### VII. METHODS OF TREATING

Certain aspects of the present disclosure provide a polypeptide for treating diseases, such as tumors. Particularly, the polypeptide may have human polypeptide sequences and thus may prevent allergic reactions in human patients, allow repeated dosing, and increase the therapeutic efficacy.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that targets CDC to cancer cells without triggering cancer cell proliferation.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

In some aspects, the disease may be, e.g., a cancer (e.g., using an agonistic antibody, such as for example an anti-CD40 agonist antibody), an infection, or an immune disease. The immune disease may be an autoimmune disease such as, e.g., lupus, rheumatoid arthritis, psoriasis, etc.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, carcinoma, lymphoma, blastoma, sarcoma, leukemia, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer, glioblastoma, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, melanoma, superficial spreading melanoma, lentigo malignant melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's macroglobulinemia), chronic lymphocytic leukemia (CLL), acute lymphoblastic leukemia (ALL), Hairy cell leukemia, multiple myeloma, acute myeloid leukemia (AML) and chronic myeloblastic leukemia.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The polypeptide may be used herein as an antitumor agent in a variety of modalities for triggering complement activation in tumor tissue or for triggering complement activation where it is considered desirable. In a particular embodiment, the invention contemplates methods of using a polypeptide as an antitumor agent, and therefore comprises contacting a population of tumor cells with a therapeutically effective amount of a polypeptide for a time period sufficient to inhibit tumor cell growth.

In one embodiment, the contacting in vivo is accomplished by administering, by intravenous intraperitoneal, or intratumoral injection, a therapeutically effective amount of a physiologically tolerable composition comprising a polypeptide of this invention to a patient. The polypeptide can be administered parenterally by injection or by gradual infusion over time. The polypeptide can be administered intravenously, intraperitoneally, orally, intramuscularly, subcutaneously, intracavity, transdermally, dermally, can be delivered by peristaltic means, or can be injected directly into the tissue containing the tumor cells.

Therapeutic compositions comprising polypeptides are conventionally administered intravenously, such as by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for initial and booster administration are also contemplated and are typified by an initial administration followed by repeated doses at one or more hour intervals by a subsequent injection or other administration. Exemplary multiple administrations are described herein and are particularly preferred to maintain continuously high serum and tissue levels of polypeptide. Alternatively, continuous intravenous infusion sufficient to maintain concentrations in the blood in the ranges specified for in vivo therapies are contemplated.

It is contemplated that a polypeptide of the invention can be administered systemically or locally to treat disease, such as to inhibit tumor cell growth or to kill cancer cells in cancer patients with locally advanced or metastatic cancers. They can be administered intravenously, intrathecally, and/or intraperitoneally. They can be administered alone or in combination with anti-proliferative drugs. In one embodiment, they are administered to reduce the cancer load in the patient prior to surgery or other procedures. Alternatively, they can be administered after surgery to ensure that any remaining cancer (e.g., cancer that the surgery failed to eliminate) does not survive.

A therapeutically effective amount of a polypeptide is a predetermined amount calculated to achieve the desired effect, i.e., to trigger CDC in the tumor tissue, and thereby mediate a tumor-ablating pro-inflammatory response. Thus, the dosage ranges for the administration of polypeptide of the invention are those large enough to produce a desired therapeutic (e.g., a reduction in cancer cell division, or an increase in cancer cell death, or other clinical benefit). The dosage preferably should not be so large as to cause significant adverse side effects, such as hyperviscosity syndromes, pulmonary edema, congestive heart failure, neurological effects, and the like. Generally, the dosage will vary with age of, condition of, sex of, and extent of the disease in the patient and can be determined by one of skill in the art. The dosage can be adjusted by the individual physician in the event of any complication. In some embodiments, the dosage may be about 0.1 mg/kg to about 10 mg/kg.

### VIII. COMBINATION THERAPY

In certain embodiments, the compositions and methods of the present embodiments involve administration of a polypeptide or antibody in combination with a second or additional therapy. Such therapy can be applied in the treatment of any disease that is responsive to CDC. For example, the disease may be cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve administering a polypeptide or antibody and a second therapy. The second therapy may or may not have a direct cytotoxic effect. For example, the second therapy may be an agent that upregulates the immune system without having a direct cytotoxic effect. A tissue, tumor, or cell can be exposed to one or more compositions or pharmacological formulation(s) comprising one or more of the agents (e.g., a polypeptide or an anti-cancer agent), or by exposing the tissue, tumor, and/or cell with two or more distinct compositions or formulations, wherein one composition provides 1) a polypeptide or antibody, 2) an anti-cancer agent, or 3) both a polypeptide or antibody and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic polypeptide or antibody and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

A polypeptide or antibody may be administered before, during, after, or in various combinations relative to an anti-cancer treatment. The administrations may be in intervals ranging from concurrently to minutes to days to weeks. In embodiments where the polypeptide or antibody is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the polypeptide and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a polypeptide or antibody is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any polypeptide or therapy of the present embodiments to a patient will follow general protocols for the administration of such compounds, taking into account the toxicity, if any, of the agents. Therefore, in some embodiments there is a step of monitoring toxicity that is attributable to combination therapy. In some embodiments involving treating a cancer in a subject, the second therapy may be, e.g., a chemotherapy, a radiotherapy, an immunotherapy (e.g., a checkpoint inhibitor, such as for example an anti-PD1 antibody or an anti-PDL1 antibody), a gene therapy, an anti-inflammatory drug, an antibiotic, or a surgery.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and suppress immune cells. Blinatumomab (Blincyto®) is such an example. Checkpoint inhibitors, such as, for example, ipilumimab, are another such example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IX. KITS

Certain aspects of the present invention may provide kits, such as therapeutic kits. For example, a kit may comprise one or more pharmaceutical composition as described herein and optionally instructions for their use. Kits may also comprise one or more devices for accomplishing administration of such compositions. For example, a subject kit may comprise a pharmaceutical composition and catheter for accomplishing direct intravenous injection of the composition into a cancerous tumor. In other embodiments, a subject kit may comprise pre-filled ampoules of a polypeptide, optionally formulated as a pharmaceutical, or lyophilized, for use with a delivery device.

Kits may comprise a container with a label. Suitable containers include, for example, bottles, vials, and test tubes. The containers may be formed from a variety of materials, such as glass or plastic. The container may hold a composition that includes a polypeptide that is effective for therapeutic or non-therapeutic applications, such as described above. The label on the container may indicate that the composition is used for a specific therapy or non-therapeutic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Library Construction Strategy for the Isolation of IgG1 Fc Domains that Bind to FcγRIIB

The mutagenesis of Fc receptor binding residues in aglycosylated V8.2 Fc was performed, and additional random mutations in Fc were introduced by error-prone PCR. FcγRIIb binding residues in Fc, which are ELLGG (233-237, EU numbering), SH (267,268), NST (297-299), and ALPAPIE (327-333), were used as site saturation mutagenesis residues for FcγRIIb-selective Fc libraries. This library was displayed on the yeast surface to screen the FcγRIIb-selective variant. N-terminus Aga2 linked Fc mutant was expressed and displayed on the cell surface of the yeast cell connected to Aga1 through two disulfide bonds. The populations which show the binding affinity to FcγRIIb were sorted with the other streptavidin-coated tetramer-FcγRs used as competitors. The first and second libraries were sorted for binding to fluorescently labeled FcγRIIb-tetramers in the presence of a high concentration of FcγRIIa_{R131}-tetramers as a competitor during the screening. To sort exclusively on a FcγRIIb-selective population, a two-step sorting method was used for third and fourth library sorting. First, the library was incubated with FcγRIIa_{R131} (the most competitive FcγR), and negative populations were sorted to remove FcγRIIa_{R131} binders. The FcγRIIa_{R131} non-binders are sorted from this step. Next, these sorted cells were incubated with FcγRIIb tetramers, and the 2b positive population was sorted. Ninety-one single clones were selected from the final library after the fourth library sorting, and 8 variants were expressed in HEK293 cells for affinity measurements.

### Example 2

### Binding Characteristics of Fc2b Variants

The Table 4 shows the list of engineered Fc variants that were used in the further experiments described below. V8.2 is a Fc variant that was generated using the methods described in Example 1. EF, V11, and V12 are published Fc variants that show enhanced binding affinity to FcγRIIb (Teige et. al., 2019) V11 and V12: (Mimoto, et al., 2013), and these variants were evaluated for comparison purposes. In the 2b18K variant, the G237D mutation is overlapping with V12 and VII. E233V is the same site but different mutation with V12 but not with V11. H268, A330 is the same site but different mutation with V12 and Vii. L328 is the same site but different mutation with EF. Substitution mutations in Table 4 are shown using Kabat numbering.

TABLE 4
Mutations of the Fc2b variants

| Variant | Mutations |
|---|---|
| WT | — |
| V8.2 | E233V, L234P, L235T, S239L, S298G, S267D, H268P, |
| | T299A, A327L, L328A, I332Q, K334V |
| 2b18K | E233V, L234D, L235F, G236R, G237D, S239L, S267D, |
| | H268P, S298G, T299A, A327L, L328A, A330H, E333I |
| 2b18KQ | E233V, L234D, L235F, G236R, G237D, S239L, S267D, |
| | H268P, R292Q, S298G, T299A, A327L, L328A, A330H, |
| | E333I |
| 2b18KQS | E233V, L234D, L235F, G236R, G237D, S239L, H268P, |
| | R292Q, S298G, T299A, A327L, L328A, A330H, E333I |
| EF | S267E, L328F |
| V11 | G237D, P238D, H268D, P271G, A330R |
| V12 | E233D, G237D, P238D, H268D, P271G, A330R |

To verify the binding affinity to Fcγreceptors, Biolayer interferometry (BLI) assays were performed on the Octet RED96 system (ForteBio Inc., California, USA). The high precision streptavidin (SAX) Biosensors (Sartorius Inc., 18-5117) were used and the assay was performed at 25° C. with shaking at 1,000 rpm. For KD measurement, the biotinylated FcgRs were immobilized onto biosensor until rich to 0.5 or 1.0 nm shift. The monoclonal antibody was associated for 2 min and dissociated for 2 min. The KD were calculated using a 1:1 binding with drifting baseline model in BIAevaluation software. Results are shown in Table 5.

TABLE 5
Binding properties of the engineered antibody variants as measured by BLI

| | FcγRI | FcγRIIa_{H131} | | FcγRIIa_{R131} | | FcγRIIb | FcγRIIIa_{F158} | FcγRIIIa_{V158} |
|---|---|---|---|---|---|---|---|---|
| Variant | (ratio of variant affinity related to WT) | | | | | | | |
| WT | 1 | 1 | | 1 | | 1 | 1 | 1 |
| V8.2 | NA | NB | | 0.35 | | 1.14 | NB | NB |
| 2b18K | NB | NB | | 0.05 | | 0.34 | NB | NB |
| 2b18KQ | NA | NB | | 0.012 | | 0.49 | NA | NB |
| 2b18KQS | NB | NB | | NB | | 0.32 | NB | NB |
| EF | NA | 0.85 | (1^{a}) | 41.1 | (864^{a}) | 37.42 (355^{a}) | NA | NA (0.1^{a}) |
| V11 | NA | 0.12 | | 0.85 | | 24.73 | NA | 0.0072 |
| V12 | NA | 0.11 | (0.1^{a}) | 1.28 | (1.5^{a}) | 68.63 (217^{a}) | NA | NA |

^{a}previously published data,
NA: not assayed,
NB: no-binding

The FcγRIIb-selective Fc variant candidates (2b18K, 2b18KQ, and 2b18KQS) showed FcγRIIb binding affinity with no or very low binding affinity to activating receptors by BLI (Table 5). In contrast, the previously reported 2b enhanced binders (i.e., EF, V11, and V12) showed binding affinity to activating receptors, especially to FcγRIIa_{R131} (FcγRIIa_{R131} has 95% identical to the extracellular domain of FcγRIIb, and only three residues located at the binding interface are different).

To measure the binding affinity of 2b18KQS Fc variant more accurately, Surface Plasmon Resonance (SPR) assay was performed with 2b18KQS variant. Results are shown in Table 6 below. Methodologies were used as described previously in Lee et al., 2017.

TABLE 6
Affinity of 2b18KQS Fc variants for Fc receptors measured by SPR (by steady state analysis)

| | | | | | | | | | FcRn |
|---|---|---|---|---|---|---|---|---|---|
| K_{DApp(nM)} | FcgRI | FcgRIIa_{H131} | FcgRIIa_{R131} | FcgRIIb | FcgRIIIa_{F158} | FcgRIIIa_{V158} | FcgRIIIb_{NA2} | C1q | (pH 5.8) |
| Her WT | 0.447 ± | 868 ± | 877 ± | 3300 ± | 2100 ± | 334 ± | 4970 ± | 11.1 ± | 548 ± |
| | 0.0202 | 6.66 | 4.1 | 3.3 | 13.3 | 0.333 | 13.3 | 4.09 | 145 |
| Her | N.B. | N.B. | 38400 ± | 14500 ± | N.B. | N.B. | N.B. | N.B. | 540 ± |
| 2b18KQS | | | 2260 | 33.3 | | | | | 186 |
| Fold | — | — | 0.023 | 0.23 | — | — | — | — | 1.01 |
| (/wt) | | | | | | | | | |

The 2b18KQS Fc variant did not display any detectable binding affinity to activating receptors and even to high affinity Fc receptor, FcγRI (Table X3). This variant showed decreased FcγRIIb binding (4-fold lower than WT Fc), and almost non-detectable binding to FcγRIIa_{R131} (40-fold lower compared to WT Fc). The binding affinity to FcRn of 2b18KQS Fc was similar to WT Fc, which may be related to circulation half-life.

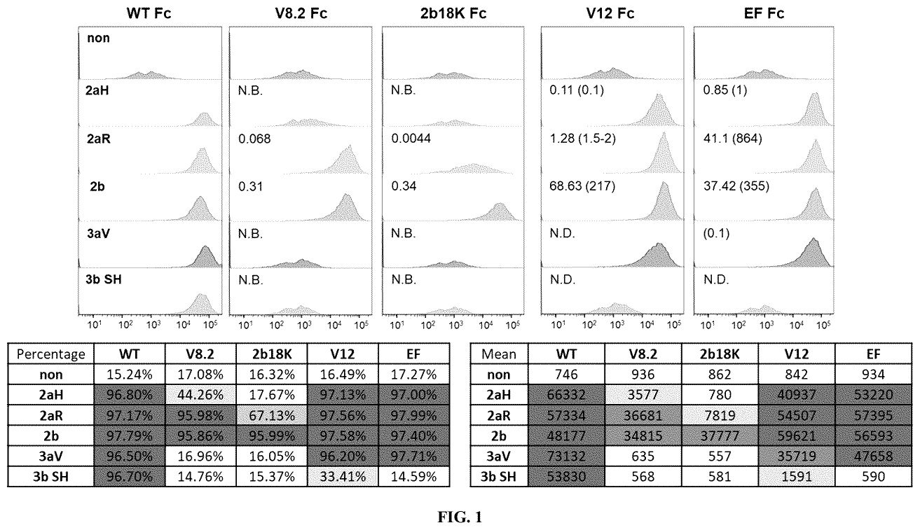

The binding characteristics of Fc variants from BLI may not always reflect the binding properties of immune complexes exactly, due to differences in avidity. In order to measure the binding profiles in high avidity conditions, opsonized SK-BR-3 and FcγR coated beads binding assay was performed with the 2b18K variant. Biotinylated Fc gamma receptors were coated on beads, to mimic geometry of Fc receptors on effector cells, and the binding was measured by flow cytometry. Results are shown in FIG. 1.

In FIG. 1, the first column shows results for WT antibody opsonized cancer cells, and the following columns show data for the V8.2, 2b18K, V12, and EF Fc variants. The numbers shown refer to the fold difference of binding affinity as compared to wild type Fc measured by BLI, and numbers in the basket (parentheses in FIG. 1) are published values from other labs. The WT shows strong binding to all Fc gamma receptors. V8.2 shows strong 2aR and 2b binding even though it shows 3-fold lower binding affinity compared to WT in BLI. 2b18K shows weak binding to 2aR and strong binding to 2b. V12 and EF show strong binding to 2a and 2b and 3aV.

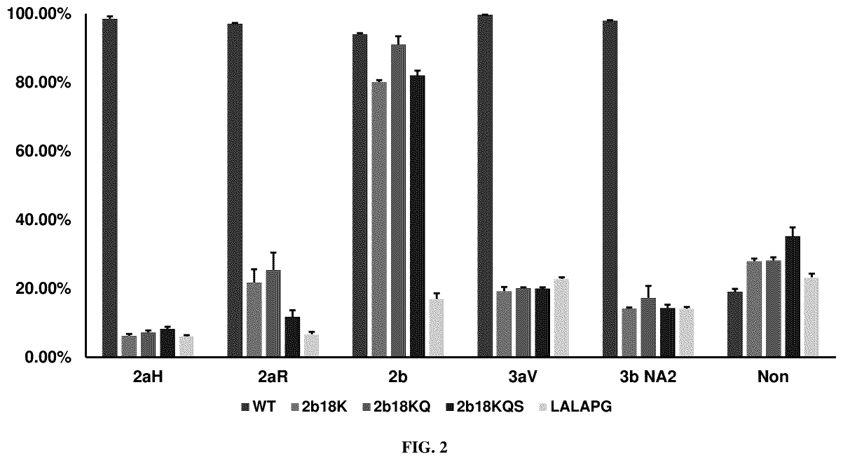

Opsonized SK-BR-3 and FcγR coated beads binding assay was performed with the 2b18KQ and 2b18KQS Fc variants (FIG. 2). The LALAPG Fc variant was included as a negative control (which is well known science Fc and completely abolish Fc mediated effector function). 2b18K variants showed no binding to FcγRIIa_{H131}, FcγRIIIa, and FcγRIIIb with maintaining their binding to FcγRIIb. 2b18KQS showed lowest binding affinity to FcγRIIa_{R131} among 2b18K variants.

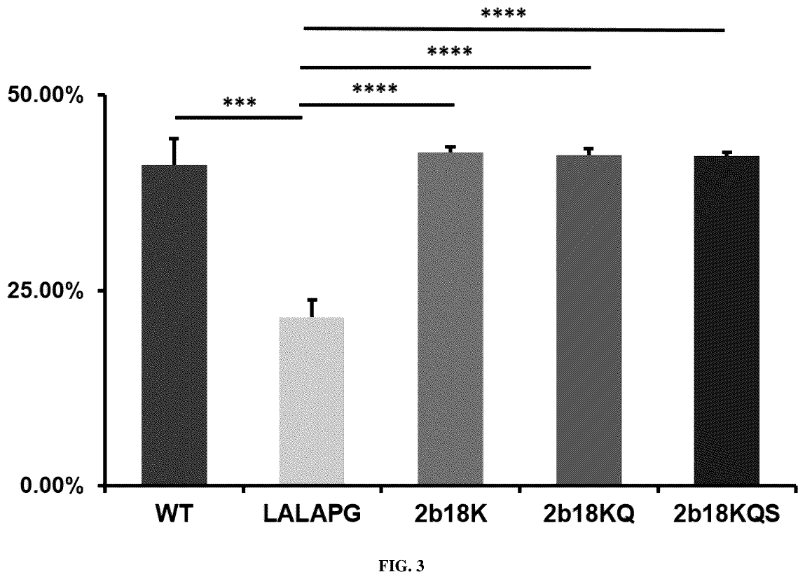

To check actual cell binding, the ICs binding assay was performed using B Burkitt's lymphoma Raji cells (ATCC CCL-86). Raji cells only express FcγRIIb among FcγRs on the surface. Thus, FcγRIIb binding was measured using B cells. Antibody coated beads were used as ICs and the binding was measured by flow cytometry. Results are shown in FIG. 3. To generate the immune complex, the 1 um fluorescence polystyrene beads were coated with antibody. These ICs were incubated with Raji cells in 4 C 1 h. The binding of ICs and Raji cells were detected by Flow cytometry.

The 2b18K variant displayed similar binding to the WT Fc and significantly better binding than the LALAPG Fc variant (FIG. 3). These results were consistent with and further supported the results obtained using opsonized SK-BR-3 and FcγRIIb coated beads binding assays.

### Example 3

### Thermostability of Fc2b Variants

The melting temperature (Tₘ) was measured to evaluate the thermodynamic stability of aglycosylated engineered Fc variants. Tₘ was measured by differential scanning fluorimetry (DSF). The Tₘ of 2b18K variants showed lower values compared to WT Fc (Table 7). These results are consistent with the idea that aglycosylation can induce a loss of thermal stability. Methods were used as described in Lee et al., 2019.

TABLE 7
Tm measurement by differential scanning fluorimetry

| Variant | Her WT | Her 2b18K | Her 2b18KQ | Her 2b18KQS |
|---|---|---|---|---|
| Tₘ(° C.) | 68.70 ± 0.17 | 57.44 ± 0.40 | 53.66 ± 0.14 | 56.28 ± 0.32 |

### Example 4

### Validation of Phagocytic Effector Function in Effector Cells

The absence of binding affinity to activating receptors in ICs diminishes the phagocytic effector function. To measure the phagocytosis rates of 2b18K variants Fc IC, the phagocytosis assay with antibody-coated pHrodo red beads was performed with THP-1 (FcγRIIa H/H homozygous) cell lines. pHrodo is a pH-sensitive dye that increases the signal at low pH. At normal pH, little or no signal is detected. The pHrodo fluorescent signal increases when the phagosome contains the target cells and becomes acidic detected on a flow cytometer. Using this dye, actual phagocytosis could be detected, and just the surface binding signal could be removed. THP-1 cells (50 k per well) were mixed in a TC-treated, round-bottom plate (Corning, 07-200-95) with antibody-coated beads (50-fold excess) in RPMI-1640 (Gibco, 11875135) and incubated for 4 hrs at 37 C 5% CO2. The cells were washed once with DPBS and immediately analyzed in a BD LSRII cytometer equipped with a high-throughput sampler. The extent of phagocytosis was expressed as the percent of bead+/pHrodo+ cells times the Dragon Green geometric MFI of the double positive population.

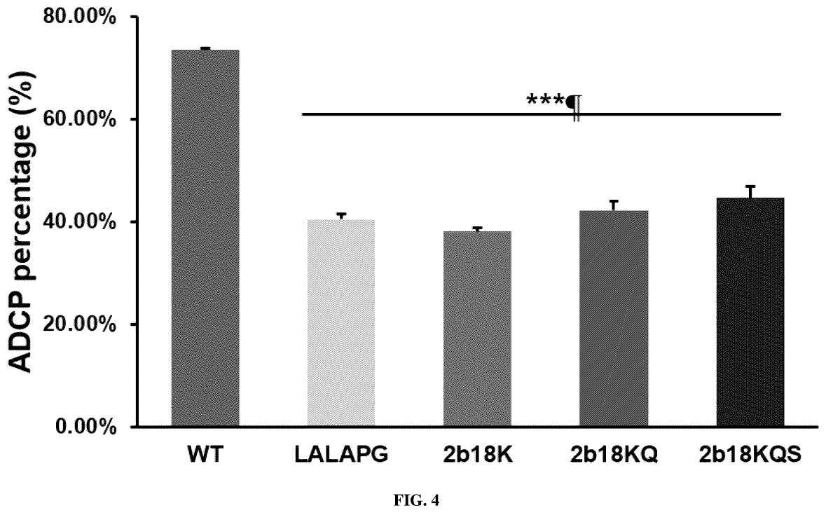

The THP-1 cells express FcγRIIaH131, which 2b18K, 2b18KQ, and 2b18KQS did not detectably bind (Table 5). The 2b18K, 2b18KQ, and 2b18KQS variants were used, and LALAPG Fc coated beads were used as a negative control. No FcγR-mediated phagocytosis was detected with 2b18K, 2b18KQ, and 2b18KQS ICs, which was consistent with the binding assay results. Results are shown in FIG. 4.

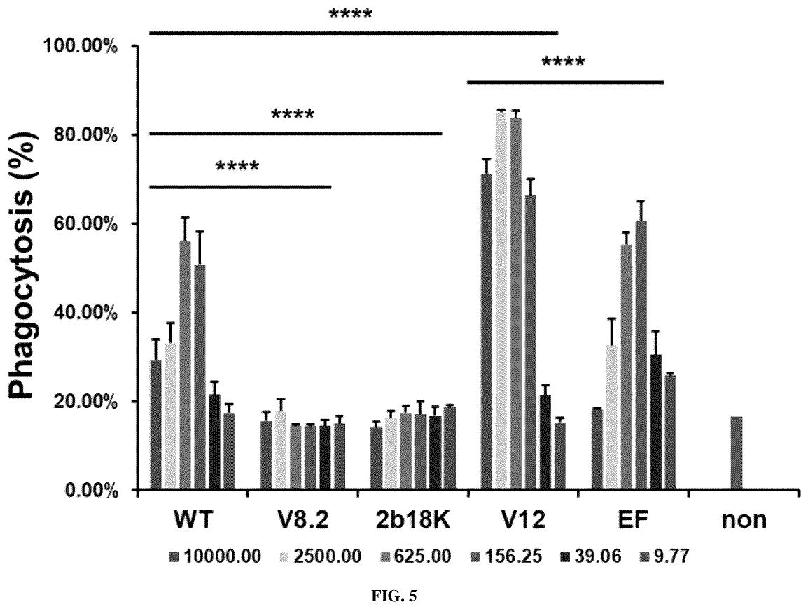

The SK-BR-3 ADCP assay with THP-1 cells was performed, revealing zero antibody mediated-phagocytosis with 2b18K and V8.2 (FIG. 5). However, high phagocytosis rates were detected in V12 and EF variants, with higher binding affinity to FcγRIIb. These results demonstrate that a higher binding affinity to FcγRIIb than activating FcγRs does not guarantee low phagocytosis activity (Table 5). The V12 variant had remarkably higher phagocytosis rates than WT and EF, even though it has a lower affinity to FcγRIIaH131. These results show that deficiency to activating receptors is indispensable to eliminating the activation of immune cells.

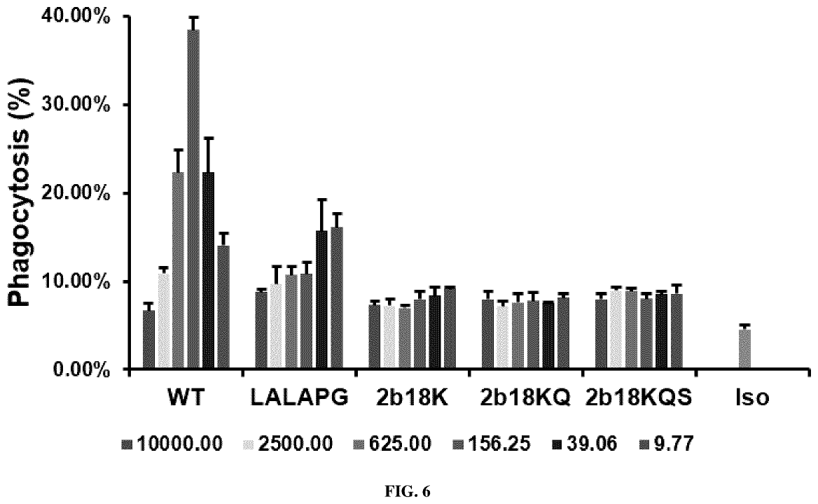

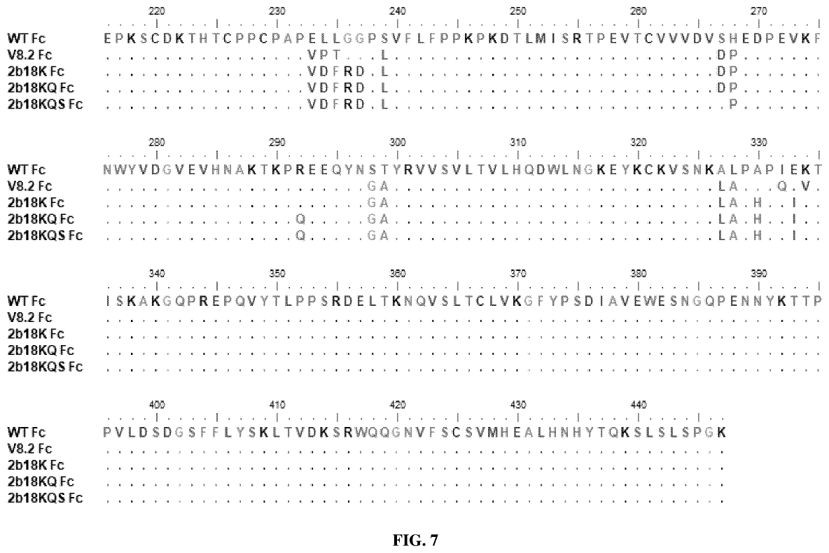

The 2b18K, 2b18KQ, and 2b18KQS variants were also tested using the SK-BR-3 ADCP assay with THP-1 cells. The ICs, which bind to only FcγRIIb, do not induce the activating FcγR and trigger the effector functions. The results are shown in FIG. 6.

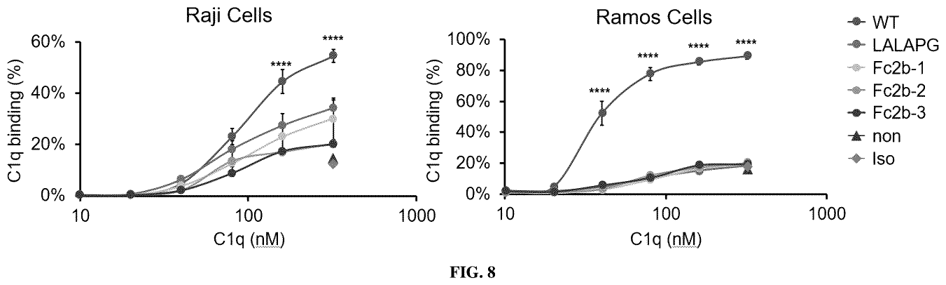

Additional experiments were performed to measure immune complex binding activities. Deposition of C1q on CD20+ Raji or Ramos cells was measured by FACS. Cells were opsonized using WT rituximab or Fc-engineered variants. All of the 2b18K, 2b18KQ, and 2b18KQS variants showed low or no C1q deposition on the cell surface. Results are shown in FIG. 8.

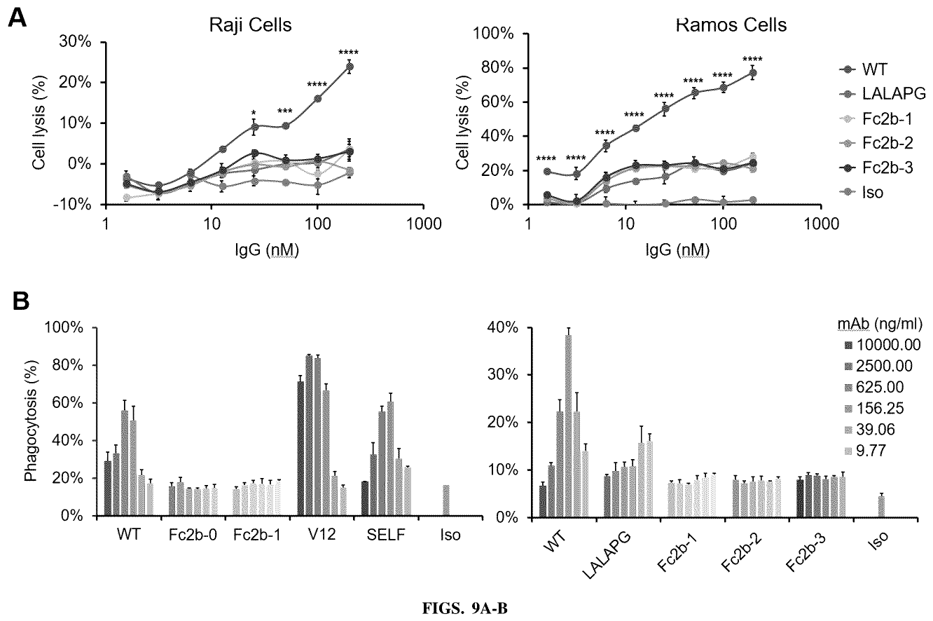

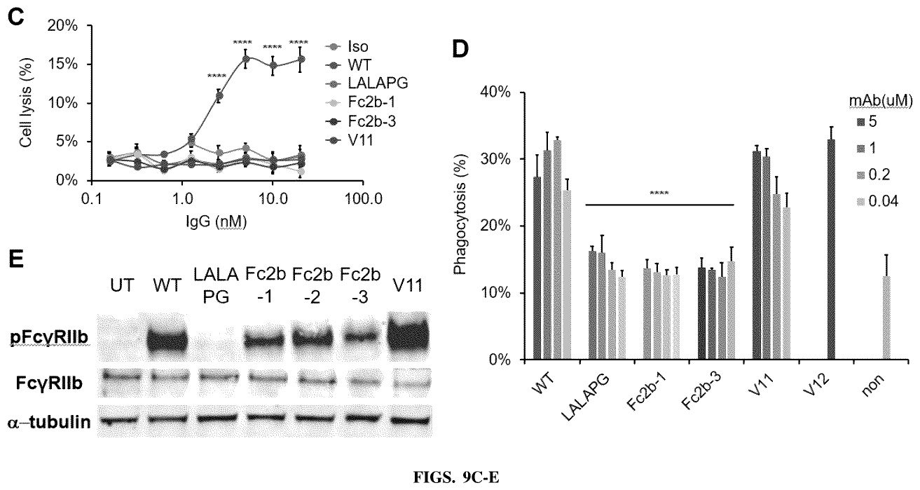

Additional experiments using in vitro cell-based assays was performed using the 2b18K, 2b18KQ, and 2b18KQS variants. No complement or activating FcγR-mediated effector functions were detected using Fc2b variants. As shown in FIG. 9A, Lysis of CD20+ Raji cells or Ramos cells by complement-dependent cytotoxicity (CDC) was measured. Cells were opsonized by various concentrations of rituximab or its Fc-engineered variants and incubated with 10% pooled human serum. Antibody-dependent cellular phagocytosis assay (ADCP) were performed with THP-1 cells as effectors and SK-BR-3 cells as targets using WT or Fc-engineered variants of trastuzumab (FIG. 9B). Antibody-dependent cellular cytotoxicity assay (ADCC) with peripheral blood mononuclear cells (PBMCs) was measured (FIG. 9C). The target cells were Raji cells opsonized with WT rituximab or its Fc-engineered variants. ADCP assay was performed with human monocyte-derived M1 macrophages as effectors and SK-BR-3 cells as targets (FIG. 9D). Various concentrations of WT trastuzumab or Fc-engineered variants were added. Phosphorylation of FcγRIIb after incubating CD20⁺ Raji cells for 1 hour with WT rituximab or Fc-engineered variants was analyzed by immunoblotting (FIG. 9E). In all assays, the Fc2b variants 2b18K, 2b18KQ, and 2b18KQS did not show any complement or activating FcγR-mediated activation, and these variants showed functional binding to FcγRIIb.

### Example 5

### Hexameric Engineered Fc Variants that Bind to FcγRIIB

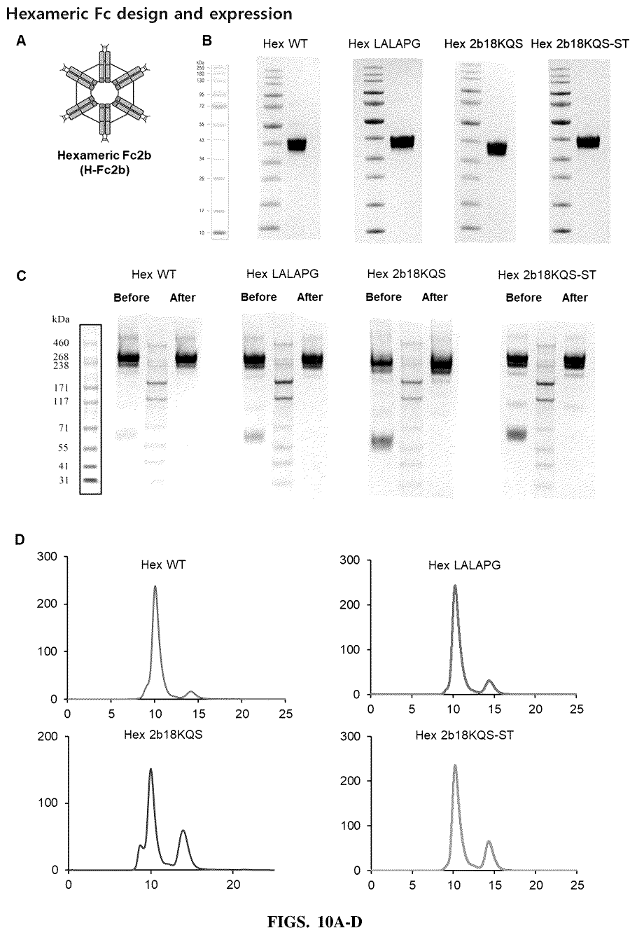

Hexameric mutant Fc regions were produced, as follows. The 18-aa residues of human IgM μ-tailpiece (PTLYNVSLVMSDTAGTCY; SEQ ID NO:6) was fused at the C terminus of human IgG1 Fc fragment (Rowley et al., 2018; Spirig et al. 2018). An extra mutation was added Leu residue at 309 (EU numbering) of the mutant IgG Fc to introduce disulfide bonds between Fcs. The engineered hexameric construct (hexameric Fc2b) was generated and results are shown in FIGS. 10A-D.

The following sequences were generated and were observed to form hexamers.

Hex 2b18KQS (aglycosylated Fc):
(SEQ ID NO: 7)
EPKSCDKTHTCPPCPAPVDFRDPLVFLFPPKPKDTLMISRTPEVTCVVV
DVSPEDPEVKFNWYVDGVEVHNAKTKPQEEQYNGAYRVVSVLTVCHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKPTLYNVSLVMSDT
AGTCY;
Hex 2b18KQS-ST (glycosylated version of 2b18KQS):
(SEQ ID NO: 8)
EPKSCDKTHTCPPCPAPVDFRDPLVFLFPPKPKDTLMISRTPEVTCVVV
DVSPEDPEVKFNWYVDGVEVHNAKTKPQEEQYNSTYRVVSVLTVCHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKPTLYNVSLVMSDT
AGTCY;
Hex2b218K (aglycosylated hexameric version of
hexameric construct of Fc variant 2b18K):
(SEQ ID NO: 12)
EPKSCDKTHTCPPCPAPVDFRDPLVFLFPPKPKDTLMISRTPEVTCVVV
DVDPEDPEVKFNWYVDGVEVHNAKTKPREEQYNGAYRVVSVLTVCHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKPTLYNVSLVMSDT
AGNCY;
and
Hex2b218K-ST (glycosylated hexameric version of
hexameric construct of Fc variant 2b18K):
(SEQ ID NO: 13)
EPKSCDKTHTCPPCPAPVDFRDPLVFLFPPKPKDTLMISRTPEVTCVVV
DVDPEDPEVKFNWYVDGVEVHNAKTKPREEQYNSTYRVVSVLTVCHQDW
LNGKEYKCKVSNKLAPHPIIKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKPTLYNVSLVMSDT
AGNCY.

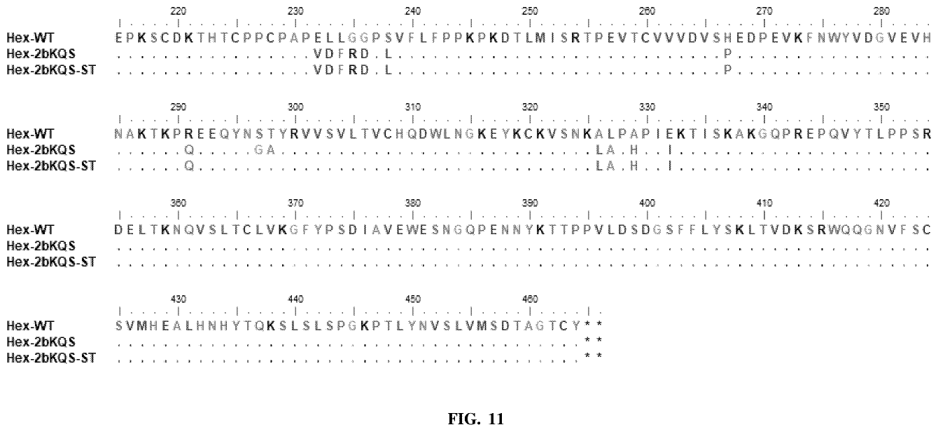

A sequence alignment showing Hexameric wild-type Fc and point mutations present in the different engineered variant Fc regions is shown in FIG. 11.

Platelet activation assay will be tested by the following methods. Platelets express the FcγRIIa on their surface, which are responsible for activating platelets when they come into contact with immune complexes. To test if there is any activation of platelets mediated by FcγRIIa in the presence of a Hexameric 2b selective Fc (Hex 2b18KQS or Hex 2b18KQS-ST), platelet activation can be assessed through co-incubation with Hexameric 2b selective Fc. It is anticipated that little or no activation of platelets will be mediated by FcγRIIa in the presence of Hexameric 2b selective Fc.

Complement dependent cytotoxicity (CDC) assay are performed as described in Lee et al. (2017). The IgG1 of Hexamer has better binding affinity to C1q. However, the hexameric Fc mediated CDC has not been previously shown. The hexameric 2b selective Fc (Hex 2b18KQS or Hex 2b18KQS-ST) were observed to have reduced C1q binding affinity, and it is anticipated that the hexameric 2b selective Fc will have no CDC activity. The CDC assay will be performed to prove there is no Hexameric 2b selective Fc mediated CDC.

B cell proliferation and differentiation assays are performed using the hexameric 2b selective Fc (Hex 2b18KQS and Hex 2b18KQS-ST). To verify the impact of sole FcγRIIb engagement on B cell proliferation and differentiation, The hexameric Fc will be treated during B cell proliferation and differentiation and analyze the impacts. B cell proliferation and differentiation assays can be performed as described in Khoenkhoen et al. (2020).

Primary monocyte and macrophage mediated ADCP assays are performed using the hexameric 2b selective Fc (Hex 2b18KQS and Hex 2b18KQS-ST). Substantial evidence indicates that blocking FcγRIIb can increase therapeutic efficacies of therapeutic antibodies. Tumor microenvironment (TME) expresses increased levels of FcγRIIb, at least partly due to the hypoxic conditions of TME. To mimic the TME, monocytes and macrophages were incubated under physiological and pharmacological hypoxic conditions, confirming the overexpression of FcγRIIb (Hussain et al., 2022). By blocking FcγRIIb, the phagocytic function was recovered significantly. The hexameric 2b selective Fc (Hex 2b18KQS and Hex 2b18KQS-ST) are tested using primary monocyte and macrophage mediated ADCP assays to measure the FcγRIIb blocking effect on monocytes and macrophages that are incubated under hypoxic conditions. Primary monocyte and macrophage mediated ADCP assays are performed as described in Kang et al. (2019).

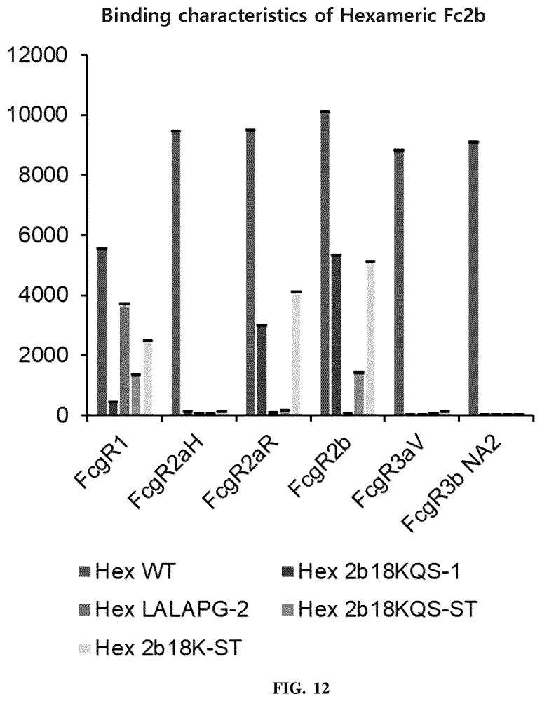

Binding characteristics of hexameric Fc2b constructs are shown in FIG. 12. FcγR coated beads and hexameric Fc binding assay results are shown using Hex 2b18KQS Fc and glycosylated Hex 2b18KQS-ST Fc. The y-axis shows the mean fluorescence intensity in FIG. 12.

The binding of hexameric Fc to Fc gamma receptors was confirmed by bead binding assay. The hexameric Fc has six valency which was provided sufficient avidity to bind with the Fc gamma receptor expressing cells or multimer of receptors. By using Fc gamma receptor coated beads, the binding characteristics of hexamer could be measured (FIG. 12). The Hex 2b18KQS showed selective binding to FcγR2b and some binding to FcγR2aR. The glycosylated hexameric 2b18KQS-ST Fc displayed increased selectivity for FcγR2b but reduced binding was observed as compared to Hex 2b18KQS. The LALAPG Fc variant was expressed as a fusion protein with the human IgM μ-tailpiece (SEQ ID NO:6) and was included as a negative control (since this Fc variant is well known science Fc and completely abolish Fc mediated effector function).

The Fc variant 2b18K was hexamerized to construct Hex 2b18K. The glycosylated variant of Hex 2b18K was also produced and is referred to as Hex 2b18K-ST and includes G298S and A299T substitution mutations in the 2b18K Fc variant. The Hex LALAPG-2 is LALAPG (L234A, L235A, P329G) variant hexameric Fc. The LALAPG Fc variant is a scilence variant that does not bind to any Fc gamma receptors, and this hexmeric variant was thus used as a negative control in experiments. Hex LALAPG-2 was generated as a hexameric Fc that was based on the LALAPG Fc variant but also contains V567I and A572G point mutations in the IgM μ-tailpiece to stabilize the structure and increase the expression yield.

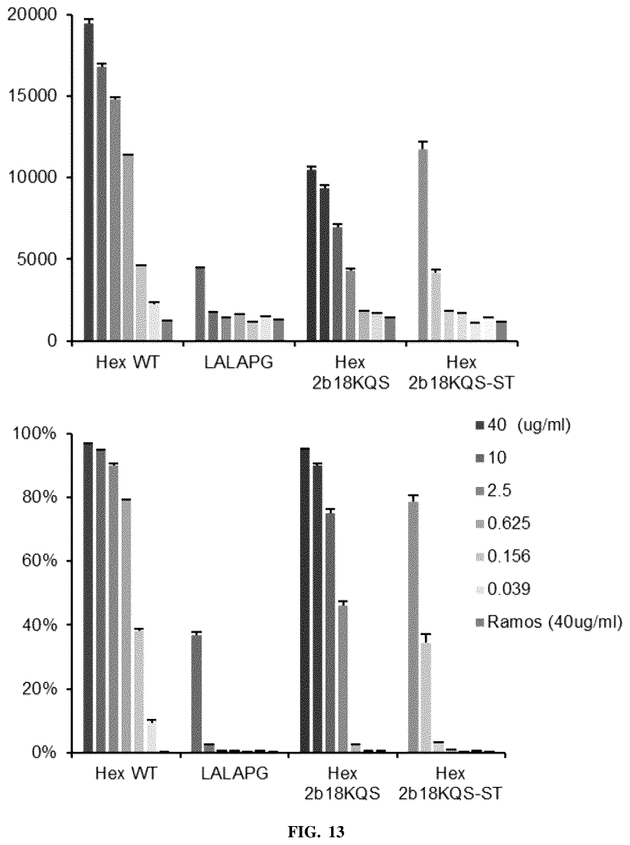

The binding of hexameric Fc to Fc gamma receptors was tested using Raji cell binding assay with antibody coated beads, and results are shown in FIG. 13. Error bars are standard error of the mean of triplicate samples. Statistical analysis was performed by one way ANOVA with Tukey s multiple comparisons test (***P 0.001, ****P 0.0001). The binding to FcγR2b was analyzed further with B cell line. The cell binding of hexameric Fc was tested with Raji cell which expresses only FcγR2b among Fc receptors (FIG. 13). The concentration dependent binding of Hex 2b18KQS Fc and glycosylated Hex 2b18KQS-ST Fc was observed.

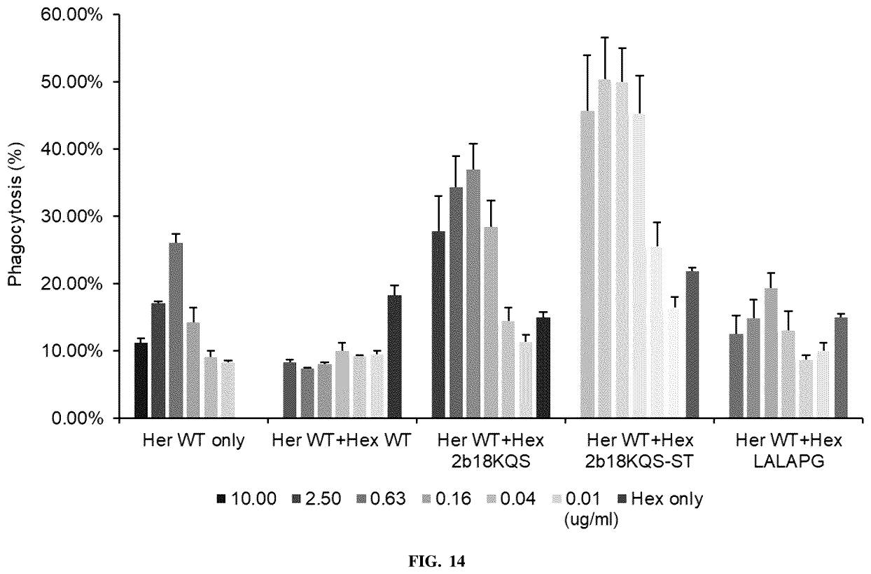

Fc gamma receptor 2b blocking by hexameric Fc were observed to increase the phagocytosis activity in monocytes, and results are shown in FIG. 14. To check the impact of FcγR2b blocking on monocyte phagocytosis, the THP-1 cell antibody-dependent cellular phagocytosis (ADCP) assay was performed with opsonized SK-BR-3 cells. Hex WT showed negligible ADCP rates because it also blocks activating Fc receptors. FcγR2b blocking by Hex 2b18KQS, and Hex 2b18KQS-ST showed increased ADCP rates compared to Her WT only.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,826,364
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,284,412
U.S. Pat. No. 4,366,241
U.S. Pat. No. 4,472,509
U.S. Pat. No. 4,498,766
U.S. Pat. No. 4,554,101
U.S. Pat. No. 4,661,913
U.S. Pat. No. 4,683,195
U.S. Pat. No. 4,683,202
U.S. Pat. No. 4,714,682
U.S. Pat. No. 4,767,206
U.S. Pat. No. 4,774,189
U.S. Pat. No. 4,800,159
U.S. Pat. No. 4,857,451
U.S. Pat. No. 4,870,287
U.S. Pat. No. 4,883,750
U.S. Pat. No. 4,938,948
U.S. Pat. No. 4,988,618
U.S. Pat. No. 4,989,977
U.S. Pat. No. 5,021,236
U.S. Pat. No. 5,160,974
U.S. Pat. No. 5,302,523
U.S. Pat. No. 5,322,783
U.S. Pat. No. 5,384,253
U.S. Pat. No. 5,464,765
U.S. Pat. No. 5,478,722
U.S. Pat. No. 5,538,877
U.S. Pat. No. 5,538,880
U.S. Pat. No. 5,550,318
U.S. Pat. No. 5,563,055
U.S. Pat. No. 5,567,326
U.S. Pat. No. 5,580,859
U.S. Pat. No. 5,589,466
U.S. Pat. No. 5,610,042
U.S. Pat. No. 5,656,610
U.S. Pat. No. 5,702,932
U.S. Pat. No. 5,736,524
U.S. Pat. No. 5,739,169
U.S. Pat. No. 5,760,395
U.S. Pat. No. 5,779,907
U.S. Pat. No. 5,780,448
U.S. Pat. No. 5,789,215
U.S. Pat. No. 5,801,005
U.S. Pat. No. 5,824,311
U.S. Pat. No. 5,824,520
U.S. Pat. No. 5,830,880
U.S. Pat. No. 5,843,650
U.S. Pat. No. 5,846,709
U.S. Pat. No. 5,846,783
U.S. Pat. No. 5,846,945
U.S. Pat. No. 5,849,497
U.S. Pat. No. 5,849,546
U.S. Pat. No. 5,849,547
U.S. Pat. No. 5,858,652
U.S. Pat. No. 5,866,366
U.S. Pat. No. 5,882,864
U.S. Pat. No. 5,889,155
U.S. Pat. No. 5,912,148
U.S. Pat. No. 5,916,776
U.S. Pat. No. 5,916,779
U.S. Pat. No. 5,922,574
U.S. Pat. No. 5,928,905
U.S. Pat. No. 5,928,906
U.S. Pat. No. 5,932,451
U.S. Pat. No. 5,935,825
U.S. Pat. No. 5,939,291
U.S. Pat. No. 5,942,391
U.S. Pat. No. 5,945,100
U.S. Pat. No. 5,981,274
U.S. Pat. No. 5,994,624
U.S. Pat. No. 7,094,571
U.S. Pat. No. 7,419,783
U.S. Pat. No. 7,611,866
U.S. Pat. No. 7,666,422
U.S. Pat. No. 7,109,304
U.S. Pat. No. 8,465,743
U.S. Pat. No. 10,526,408
U.S. Patent Publ. 2003/0180937
U.S. Patent Publ. 2003/0219870
U.S. Patent Publ. 2005/0260736
U.S. Patent Publ. 2006/0173170
U.S. Patent Publ. 2008/0292646
U.S. Patent Publ. 2009/0304666
PCT Appln. PCT/US87/00880
PCT Appln. PCT/US89/01025
PCT Appln. WO 88/10315
PCT Appln. WO 89/06700
PCT Appln. WO 90/07641
PCT Appln. WO 93/06213
PCT Appln. WO 94/09699
PCT Appln. WO 95/06128
PCT Appln. WO 2008/137475
PCT Appln. WO2005/018572A2
PCT Appln. WO 2012/031744
PCT Appln. WO 2012/079000
PCT Appln. WO 2013/059593
PCT Appln. WO 2012/115241 A1
Abbondanzo et al., Breast Cancer Res. Treat., 16:182(151), 1990.
Ahmed et al., Clinical Cancer Research, 16(2):474-485, 2010.
Ahouse et al., J. Immunol., 151:6076-6088, 1993.
Allen and Seed, Nucleic Acids Res., 16:11824, 1988.
Andersen et al., Eur. J. Immunol., 36:3044-3051, 2006.
Antibodies: A Laboratory Manual, Cold Spring Harbor Laboratory, 1988
Atherton et al., Biol. Reprod., 32(1):155-171, 1985.
Austin-Ward and Villaseca, Revista Medica de Chile, 126(7):838-845, 1998.
Ausubel et al., In: Current Protocols in Molecular Biology, John, Wiley & Sons, Inc, N Y, 1994.
Baneyx and Mujacic, Nat. Biotechnol., 22:1399-1408, 2004.
Barrington, R. A. et al., J. Exp. Med. 196, 1189-1199, 2002.
Bellus, J. Macromol. Sci. Pure Appl. Chem., A31(1): 1355-1376, 1994.
Berberian et al., 1993.
Berntzen et al., J. Immunol. Methods, 298:93-104, 2005.
Better et al., Science, 240: 1041-10433, 1988.
Bocek and Pecht, FEBS Lett., 331, 86-90, 1993.
Boeke et al., Mol. Gen. Genet., 186, 1982.
Bolland and Ravetch, Adv. Immunol., 72:149-177, 1999.
Borrok et al., ACS Chem. Biol., 7:1596-1602, 2012.
Boruchov et al., J. Clin. Invest., 115:2914-2923, 2005.
Boss et al., Nucleic Acids Res., 12:3791-3806, 1984.
Bowden and Georgiou, J. Biol. Chem., 265:16760-16766, 1990.
Bukau et al., J. Bacteriol., 163:61, 1985.
Bukowski et al., Clinical Cancer Res., 4(10):2337-2347, 1998.
Burman et al., J. Bacteriol., 112:1364, 1972.
Burton and Burton, Nat. Rev. Immunol., 4:89-99, 2004.
Cabilly et al., Proc. Natl. Acad. Sci. USA, 81:3273-3277, 1984.
Carbonelli et al., FEMS Microbiol Lett., 177:75-82. 1999
Chames et al., Proc. Natl. Acad. Sci. USA, 97:7969-7974, 2000.
Chen and Guillemin, Int J Tryptophan Res, 2:1-19, 2009.
Chen and Okayama, Mol. Cell Biol., 7(8):2745-2752, 1987.
Choi et al. PLoS One.; 10(12): e0145349, 2015.
Christodoulides et al., Microbiology, 144(Pt 11):3027-3037, 1998.
Chu et al., 2008.
Cleary et al., 1994.
Cocea, Biotechniques, 23(5):814-816, 1997.
Collins et al., Immunogenetics, 45:440-443, 1997.
Curran et al., Proceedings of the National Academy of Sciences, 107:4275-4280, 2010.
Daeron, Annu. Rev. Immunol., 15:203-234, 1997.
Dall'Acqua et al., J. Immunol., 177:1129-1138, 2006.
Daugherty et al., Protein Eng., 12:613 621, 1999.
Davidson et al., J. Immunother., 21(5):389-398, 1998.
De Jager et al., Semin. Nucl. Med., 23(2):165-179, 1993.
de Jong et al., Int J Gynecol Cancer, 21(7):1320-1327, 2011.
de Kruif and Logtenberg, J. Biol. Chem., 271:7630-7634, 1996.
Decad and Nikaido, J. Bacteriol., 128:325, 1976.
Della Chiesa et al., Blood, 108(13):4118-4125, 2006.
Desai et al., Cancer Res., 58:2417-2425, 1998.
Dholakia et al., J. Biol. Chem., 264(34):20638-20642, 1989.
Diebolder et al., Science, 343:1260-1263, 2014.
Doolittle and Ben-Zeev, Methods Mol Biol, 109:215-237, 1999.
Edelman et al., Proc Natl Acad Sci USA, 63:78-85, 1969.
Eigenbrot et al., J. Molec. Biol., 229:969-995, 1993.
Elbein et al., Glycobiology, 13:17R-27, 2003.
Elvin et al., Int. J. Pharm., 440:83-98, 2013.
European Appln. 320 308
European Appln. 329 822
Fahnestock et al., J. Bacteriol., 167:870-880, 1986.
Farmer et al., FEMS Microbiol. Lett., 176:11, 1999.
Fechheimer, et al., Proc Natl. Acad. Sci. USA, 84:8463-8467, 1987.
Fraley et al., Proc. Natl. Acad. Sci. USA, 76:3348-3352, 1979.
Francisco et al., Proc. Natl. Acad. Sci. USA, 90:10444-10448, 1993.
Frohman, In: PCR Protocols: A Guide To Methods And Applications, Academic Press, N.Y., 1990.
Fromant et al., Anal. Biochem., 224:347-353, 1995.
Gaboriaud et al., J. Biol. Chem., 278:46974-46982, 2003.
Garinot-Schneider et al., J. Mol. Biol., 260:731-742, 1996.
GB Appln. 2 202 328
Georgiou and Segatori, Current Opin. Biotech., 16:538-545, 2005.
Ghetie and Ward, Annu. Rev. Immunol., 18:739-766, 2000.
Godin-Ethier et al., Clinical Cancer Research, 17(22):6985-6991, 2011.
Golay et al., Blood, 95:3900-3908, 2000.
Gomi et al., J. Immunol., 144:4046-4052, 1990.
Gopal, Mol. Cell Biol., 5:1188-1190, 1985.
Graham and Van Der Eb, Virology, 52:456-467, 1973.
Griffiths and Duncan, Curr Opin. Biotechnol., 9:102-108, 1998.
Guddat et al., Proc. Natl. Acad. Sci. USA, 90:4271-4275, 1993.
Gulbis and Galand, Hum. Pathol., 24(12):1271-1285, 1993.
Guzman et al., J. Bacteriol., 177:4121-4130, 1995.
Halloran et al., J. Immunol., 153:2631-2641, 1994.
Hanibuchi et al., Int. J. Cancer, 78(4):480-485, 1998.
Harkki et al., BioTechnology, 7:596-603, 1989.
Harland and Weintraub, J. Cell Biol., 101(3):1094-1099, 1985.
Harvey et al., J. Immunol. Methods. 308:43-52, 2006.
Harvey et al., Proc. Natl. Acad. Sci. USA, 101:9193-9198, 2004.
Hayhurst et al., J. Immunol. Methods, 276:185-196, 2003.
Hellstrand et al., Acta Oncologica, 37(4):347-353, 1998.
Hobot et al., J. Bacteriol., 160:143, 1984.
Hoey et al., Exp Biol Med (Maywood) 244(17): 1568-1576, 2019.
Hollander, Front. Immun., 3:3, 2012.
Holmgaard et al., The Journal of Experimental Medicine, 210:1389-1402, 2013.
Hoogenboom and Winter, J. Mol. Biol., 227:381-388, 1992.
Hoogenboom et al., Immunotechnology, 4:1-20, 1998.
Hoover and Lubkowski, Nucl. Acids Res., 30:e43, 2002.
Hopwood et al., In: Genetic Manipulation of Streptomyces, A Laboratory Manual, The John Innes Foundation, Norwich, Conn., 1985.
Horton et al., J. Immunol., 186(7):4223-4233, 2011.
Hughes-Jones and Gardner, Mol. Immunol., 16:697-701, 1979.
Hui and Hashimoto, Infection Immun., 66(11):5329-5336, 1998.
Hussain et al., Journal of Experimental & Clinical Cancer Research 2022 41:1 41, 1-32, 2022.
Idusogie et al., J. Immunol., 166:2571-2575, 2001.
Innis et al., Proc. Natl. Acad. Sci. USA, 85(24):9436-9440, 1988.
Irvin et al., J. Bacteriol., 145:1397, 1981.
Ito et al., J. Biochem., 79:1263, 1976.
Janeway et al., Immunobiology: The Immune System in Health and Disease. 6th Edition. New York: Garland Publishing, 2005.
Jefferis, Adv. Exp. Med. Biol., 564:143-148, 2005.
Jefferis, Biotechnol. Prog., 21:11-16, 2005.
Jeong and Lee, Appl. Environ. Microbiol., 69:1295-1298, 2003.
Jouenne and Junter, FEMS Microbiol. Lett., 56:313, 1990.
Jung et al., ACS Chem. Biol., 8:368-375, 2012.
Jung et al., Biotechnol Bioeng, 98:39-47, 2007
Jung et al., Proc. Natl. Acad. Sci. USA, 107:604-609, 2010.
Jung et al., Protein Expr. Purif, 31:240-246, 2003.
Kabat et al., In: Sequences of Proteins of Immunological Interest, U.S. Dept. of Health and Hum. Serv., Bethesda, 1991.
Kaeppler et al., Plant Cell Reports, 9:415-418, 1990.
Kalergis and Ravetch, J. Exp. Med., 195:1653-1659, 2002.
Kaneda et al., Science, 243:375-378, 1989.
Kang et al., 1988.
Kang et. al., Front Immunol. 2019 Mar. 27; 10:562, 2019.
Kaper et al., PLoS Biology, 5(10):e257, 2007.
Kato et al, J. Biol. Chem., 266:3361-3364, 1991.
Kawarasaki et al., Nucleic Acids Res., 31:e126, 2003.
Kelton et al., Chemistry & Biology, 21(12): 1603-1609, 2014.
Khatoon et al., Ann. Neurol, 26(2):210-215, 1989.
Khoenkhoen et. al., Front Immunol. 2020; 11:571321. Published Sep. 29. 2020.
Kim et al., Eur. J. Immunol., 24:2429-2434, 1994.
King et al., J. Biol. Chem., 264(17):10210-10218, 1989.
Kipriyanov and Little, Mol. Biotechnol., 12:173-201, 1999.
Kjaer et al., FEBS Lett., 431:448-452, 1998.
Knight et al., Mol. Immunol., 32:1271-1281, 1995.
Kohler and Milstein, Nature, 256:495-497, 1975.
Kohler et al., 1989
Kouzarides and Ziff, Nature, 336:646-6451, 1988.
Kreier et al., 1991.
Kuroda et al., Lancet., 357:1225-1240, 2001.
Kwoh et al., Proc. Natl. Acad. Sci. USA, 86:1173, 1989.
Kyte and Doolittle, 1982.
Labischinski et al., J. Bacteriol., 162:9, 1985.
Landschulz et al., Science, 240:1759-1764, 1988.
Lanio and Jeltsch, Biotechniques, 25:962-955, 1998.
Lazar et al., Proc. Natl. Acad. Sci. USA, 103:4005-4010, 2006.
Lee et. al., Nat Immunol. 18, 889-898, 2017.
Lee, et. al., Nat Commun 10, 5031, 2019.
Lee, et. al., Nat. Immunol. 18, 889-898, 2017.
Lei et al., J. Bacteriol., 169:4379-4383, 1987.
Lenert et al., 1990.
Levenson et al., Hum. Gene Ther., 9(8):1233-1236, 1998.
Leysath et al., J. Mol. Biol., 387:680-693, 2009.
Li et al., J. Mol. Biol., 337:743-759, 2004.
Lipowska-Bhalla et al., Cancer Immunology Immunotherapy, 61(7):953-962, 2012.
Lob et al., Nat Rev Cancer, 9(6):445-452, 2009.
Lordanescu, J. Bacteriol, 12:597 601, 1975.
Mandi and Vecsei, J Neural Transm, 119(2):197-209, 2012.
Maniatis, et al., Molecular Cloning, A Laboratory Manual, Cold Spring Harbor Press, Cold Spring Harbor, N.Y., 1988.
Marciano et al., Science, 284:1516, 1999.
Masaki et al., Nucleic Acids Res., 13:1623-1635, 1985.
Mazor et al., Nat. Biotech., 25(5):563-565, 2007.
Mellor et al., Gene, 24:1-14, 1983.
Mezrich et al., The Journal of Immunology, 185(6):3190-3198, 2010.
Michaelsen et al., Scand. J. Immunol., 70:553-564, 2009.
Mimoto et al., Protein Eng. Des. Sel. 26, 589-598, 2013.
Moore et al., mAbs, 2:181-189, 2010.
Munson and Pollard, Anal. Biochem., 107:220, 1980.
Nagaoka and Akaike, Protein Engineering, 16: 243-245, 2003.
Natsume et al., Cancer Res., 68:3863-3872, 2008.
Nicolau and Sene, Biochim. Biophys. Acta, 721:185-190, 1982.
Nicolau et al., Methods Enzymol., 149:157-176, 1987.
Nikaido and Nakae, Adv. Microb. Physiol., 20:163, 1979.
Nikaido and Vaara, Microbiol. Rev., 49:1, 1985.
Nikaido, J. Bacteriology, 178(20):5853-5859, 1996.
Nimmerjahn F. and Ravetch JV, Science. 310(5753):1510-1512, 2005.
O'Brien et al., Protein Expr. Purif., 24:43-50, 2002.
Ober et al., J. Immunol., 172:2021-2029, 2004b.
Ober et al., Proc. Natl. Acad. Sci. USA, 101:11076-11081, 2004a.
Olsson et al., Eur. J. Biochem., 168:319-324, 1987.
Opitz et al., Nature, 478(7368):197-203, 2011.
Opitz et al., PLoS One, 6(5):e19823, 2011.
Orlandi et al., Proc. Natl. Acad. Sci. USA, 86:3833-3837, 1989.
Osborn et al., J. Biol. Chem, 247:3973-3986, 1972.
Owens and Haley, Biochem. Biophys. Res. Commun., 142(3):964-971, 1987.
Painbeni et al., Proc Natl. Acad. Sci. USA, 94:6712, 1997.
Pavlou and Belsey, Eur. J. Pharm. Biopharm., 59:389-396, 2005.
Penttila et al., Gene, 61:155-164, 1987.
Phillips, N. E. & Parker, D. C. J. Immunol. 132, 627-632, 1984.
Pilotte et al., Proc Natl Acad Sci USA, 109(7):2497-2502, 2012.
Potrykus et al., Mol. Gen. Genet., 199(2):169-177, 1985.
Potter and Haley, Methods Enzymol, 91:613-633, 1983.
Prendergast, Nature, 478(7368):192-194, 2011.
Purvis et al., Appl. Environ. Microbiol., 71:3761-3769, 2005.
Qin et al., Proc. Natl. Acad. Sci. USA, 95(24):14411-14416, 1998.
Qin, D. et al. J. Immunol. 164, 6268-6275, 2000.
Raghavan and Bjorkman, Annu. Rev. Cell Dev. Biol., 12:181-220, 1996.
Ramsland et al., J. Immunol., 187:3208-3217, 2011.
Rankin et al., Blood 108: 2384-2391, 2006.
Rao and Torriani, J. Bacteriol., 170, 5216, 1988.
Ravetch and Perussia et al., J. Exp. Med., 170:481-497, 1989.
Ravetch et al., Science, 234:718-725, 1986.
Remington's Pharmaceutical Sciences, 18th Ed. Mack Printing Company, 1289-1329, 1990.
Rippe, et al., Mol. Cell Biol., 10:689-695, 1990.
Rodewald, J. Cell Biol., 71:666-669, 1976.
Rowley et al., Communications Biology 2018 1:1 1, 1-12, 2018.
Ruhlmann et al., FEBS Lett., 235:262-266, 1988.
Rutella et al., Endocr Metab Immune Disord Drug Targets, 9(2):151-177, 2009.
Sambrook et al., In: Molecular cloning: a laboratory manual, 2^{nd} Ed., Cold Spring Harbor Laboratory Press, Cold Spring Harbor, N Y, 1989.
Sasso et al., 1989.
Sazinsky et al., Proc. Natl. Acad. Sci. USA, 105:20167-20172, 2008.
Schellenberger et al., Nature Biotech., 27:1186-1190, 2009.
Schierle et al., J. Bacteriol., 185:5706-5713, 2003.
Schneider and Zacharias, Mol. Immunol., 51:66-72, 2012.
Sears et al., J. Immunol., 144:371-378, 1990.
Sergina and Moasser, Trends in Molec. Med., 13:527-534, 2007.
Shields et al., J. Biol. Chem., 276:6591-6604, 2001.
Shin et al., Proc Natl Acad Sci USA, 110(30):12391-12396, 2013.
Shorki et al., 1991.
Shuttleworth et al., Gene, 58(2-3):283-295, 1987.
Sibakov et al., Eur. J. Biochem., 145:567 572, 1984.
Sidman, C. L. and Unanue, E. R. J. Exp. Med. 144, 882-896, 1976.
Silvermann et al., 1995.
Simister and Mostov, Nature, 337(6203):184-187, 1989.
Sinclair, N. R. J. Exp. Med. 129, 1183-1201, 1969.
Sledge and Bing, J. Biol. Chem., 248:2818-2823, 1973.
Sondermann et al., J. Mol. Biol., 309:737-749, 2001.
Song et al., International Immunopharmacology, 11(8):932-938, 2011.
Spirig et al., The Journal of Immunology 200, 2542-2553, 2018.
Stenberg et al., Mol. Microbiol., 6:1185-1194, 1992.
Stengelin et al., Embo J, 7:1053-1059, 1988.
Stone et al., ACS Chemical Biology, 5:333-342, 2010.
Stuart et al., Embo J., 8:3657-3666, 1989.
Stuart et al., J. Exp. Med., 166:1668-1684, 1987.
Teige et. al., Front. Immunol. 0, 481, 2019.
Tominaga et al., Biochem. Biophys. Res. Commun., 168:683-689, 1990.
Uhlen et al., J. Biol. Chem., 259:1695-702, 1984.
Van Wielink and Duine, Trends Biochem Sci., 15:136, 1990.
Wada et al., J. Biol. Chem., 274:17353-17357, 1999.
Walker et al., Nucleic Acids Res., 20(7):1691-1696, 1992.
Walport, Complement. First of two parts. N. Engl. J. Med., 344:1058-1066, 2001.
Walport, Complement. Second of two parts. N. Engl. J. Med., 344:1140-1144, 2001.
Ward, Proc, Embo-Alko Workshop on Molecular Biology of Filamentous Fungi, Helsinki, 119-128, 1989.
Wawrzynczak and Thorpe, Vogel (Ed.), NY, Oxford University Press, 28, 1987.
Wilson et al., Cancer Cell, 19(1):101-113, 2011
Wong et al., Gene, 10:87-94, 1980.
Wozniak-Knopp et al., Protein Engineering, Design and Selection 23(4): 289-297, 2010.
Wright and Morrison, Trends Biotechnol., 15:26-32, 1997.
Yao et al., Mol Biosyst, 7(9):2608-2614, 2011.
Yoshikawa et al., Eur J Haematol, 84(4):304-309, 2010.
Yuan, et al., 2022.
Zeger et al., Proc. Natl. Acad. Sci. USA, 87:3425-3429, 1990.
Zhang et al., Immunogenetics, 39:423-437, 1994.
Zhang et al., Microbiology, 144(Pt 4):985-991, 1998.
Zohair et al., Biochem. J., 257:865-873, 1989.

What is claimed is:

1. A polypeptide comprising a mutant or variant human IgG Fc domain capable of binding human FcγRIIb,
wherein the mutant or variant human IgG Fc domain comprises substitution mutations of valine at position 233 (E233V), leucine at position 239 (S239L), proline at position 238 (H268P), leucine at position 327 (A327L), alanine at position 328 (L328A), and substitution mutations at positions 234 (L234) and 235 (L235);
with amino acid position numbering being according to the Kabat system.

2. The polypeptide of claim 1, wherein the mutant or variant human IgG Fc domain further comprises substitution mutations of glycine at position 298 (S298G) and alanine at position 299 (T299A).

3. The polypeptide of any one of claims 1-2, wherein the substitution mutation at position 234 is proline at position 234 (L234P) or aspartic acid at position 234 (L234D).

4. The polypeptide of claim 3, wherein the substitution mutation at position 234 is aspartic acid at position 234 (L234D).

5. The polypeptide of any one of claims 1-4, wherein the substitution mutation at position 235 is threonine at position 235 (L235T) or phenylalanine at position 235 (L235F).

6. The polypeptide of claim 5, wherein the substitution mutation at position 235 is phenylalanine at position 235 (L235F).

7. The polypeptide of claim 1, wherein the substitution mutation at position 234 is proline at position 234 (L234P), and wherein the substitution mutation at position 235 is threonine at position 235 (L235T).

8. The polypeptide of claim 7, wherein the mutant or variant human IgG Fc domain further comprises a substitution mutation of aspartic acid at position 237 (S267D).

9. The polypeptide of claim 8, wherein the mutant or variant human IgG Fc domain further comprises a substitution mutations of glutamine at position 332 (I332Q) and/or valine at position 334 (K334V).

10. The polypeptide of claim 9, wherein the mutant or variant human IgG Fc domain comprises or consists of Fc V8.2 (SEQ ID NO:2).

11. The polypeptide of claim 1, wherein the substitution mutation at position 234 is aspartic acid at position 234 (L234D), and wherein the substitution mutation at position 235 is phenylalanine at position 235 (L235F).

12. The polypeptide of claim 11, wherein the mutant or variant human IgG Fc domain further comprises 1, 2, 3, or all of: substitution mutations arginine at position 236 (G236R), aspartic acid at position 237 (G237D), histidine at position 330 (A330H), and/or isoleucine at position 333 (E333I).

13. The polypeptide of claim 12, wherein the mutant or variant human IgG Fc domain further comprises a substitution mutation of aspartic acid at position 267 (S267D).

14. The polypeptide of claim 13, wherein the mutant or variant human IgG Fc domain comprises or consists of Fc 2B18K (SEQ ID NO:3).

15. The polypeptide of claim 13, wherein the mutant or variant human IgG Fc domain further comprises a substitution mutation of glutamine at position 292 (R292Q).

16. The polypeptide of claim 15, wherein the mutant or variant human IgG Fc domain comprises or consists of Fc 2B18KQ (SEQ ID NO:4).

17. The polypeptide of claim 12, wherein the mutant or variant human IgG Fc domain further comprises a substitution mutation of glutamine at position 292 (R292Q).

18. The polypeptide of claim 17, wherein the mutant or variant human IgG Fc domain comprises or consists of Fc 2B18KQS (SEQ ID NO:5).

19. The polypeptide of any one of claims 1-18, wherein the mutant or variant human IgG Fc domain is aglycosylated.

20. The polypeptide of any one of claims 1-18, wherein the mutant or variant human IgG Fc domain is glycosylated.

21. The polypeptide of any one of claims 1-20, wherein the Fc domain does not selectively or detectably bind to 1, 2, 3, 4, or all of: a human FcγRI, FcγRIIa H131, FcγRIIIa F158, FcγRIIIa V158, and/or C1q polypeptide.

22. The polypeptide of claim 21, wherein the Fc domain does not selectively or detectably bind to a human FcγRI.

23. The polypeptide of claim 22, wherein the Fc domain does not selectively or detectably bind to any of human FcγRIIa H131, FcγRIIIa F158, and FcγRIIIa V158.

24. The polypeptide of any one of claims 1-23, wherein the Fc domain has a binding to FcγRIIa_{R131} that is at least 30-fold or 40-fold less than wild-type Fc binding.

25. The polypeptide of any one of claims 3-24, wherein the Fc domain further comprises a substitution mutation at position 298 and/or a substitution mutation at position 299.

26. The polypeptide of claim 25, wherein the Fc domain comprises a leucine at amino acid position 299 (T299L).

27. The polypeptide of any one of claims 3-9, 11-13, 15, 17, or 19-24, wherein the Fc domain comprises serine at position 298 and threonine position 299.

28. The polypeptide of any one of claims 1-27, further comprising a non-Fc receptor (non-FcR) binding domain.

29. The polypeptide of claim 28, wherein the non-FcR binding domain is an Ig variable domain, an antibody variable domain, or an antibody heavy chain variable domain.

30. The polypeptide of claim 29, wherein the polypeptide is a full-length antibody.

31. The polypeptide of claim 30, wherein the antibody is an agonistic antibody.

32. The polypeptide of claim 31, wherein the agonistic antibody is an anti-CD40 agonist antibody.

33. The polypeptide of claim 29, wherein the Ig variable domain comprises an antibody heavy chain variable domain.

34. The polypeptide of claim 33, wherein the Ig variable domain is comprised in a single domain antibody.

35. The polypeptide of claim 29, wherein the Ig variable domain comprises an ScFv.

36. The polypeptide of any one of claims 1-35, wherein the mutant or variant human IgG Fc domain is comprised in a multimeric oligomer.

37. The polypeptide of claim 36, wherein the multimeric oligomer is further defined as a hexameric Fc fusion protein.

38. The polypeptide of claim 37, wherein the hexameric Fc fusion protein comprises
(SEQ ID NO: 6)
PTLYNVSLVMSDTAGTCY,
(SEQ ID NO: 9)
PTLYNVSLIMSDTGGTCY,
(SEQ ID NO: 10)
PTLYNVSLIMSDTAGTCY,
or
(SEQ ID NO: 11)
PTLYNVSLVMSDTGGTCY.

39. The polypeptide of claim 38, wherein the hexameric Fc fusion protein comprises
(SEQ ID NO: 6)
PTLYNVSLVMSDTAGTCY.

40. The polypeptide of any one of claims 37-39, wherein the mutant or variant human IgG Fc domain comprises a substitution mutation of Leu residue at position 309, with amino acid position numbering being according to the Kabat system.

41. The polypeptide of any one of claims 37-40, wherein the hexameric Fc fusion protein is glycosylated.

42. The polypeptide of any one of claims 37-40, wherein the hexameric Fc fusion protein is aglycosylated.

43. The polypeptide of any one of claims 37-42, wherein the mutant or variant human IgG Fc domain comprises a Gly residue at position 298 and/or an Ala residue at position 299.

44. The polypeptide of claim 37-43, wherein the mutant or variant human IgG Fc domain comprises a Ser residue at position 298 and/or a Thr residue at position 299.

45. The polypeptide of claim 37, wherein the hexameric Fc fusion protein comprises SEQ ID NO:7, SEQ ID NO:8, SEQ ID NO:12, or SEQ ID NO:13.

46. The polypeptide of claim 45, wherein the hexameric Fc fusion protein comprises SEQ ID NO:7 or SEQ ID NO:8.

47. The polypeptide of any one of claims 1-46, wherein the polypeptide or antibody is chemically conjugated to or covalently bound to a toxin.

48. The polypeptide of claim 28, wherein the non-FcR binding region binds a cell-surface protein.

49. The polypeptide of claim 28, wherein the non-FcR binding region binds a soluble protein.

50. The polypeptide of claim 28, wherein the non-FcR binding domain comprises a single domain antibody, a scFv, or a nanobody.

51. The polypeptide of any one of claims 1-50, wherein the polypeptide is aglycosylated.

52. The polypeptide of any one of claims 1-50, wherein the polypeptide is glycosylated.

53. The polypeptide of any one of claims 1-52, wherein the Fc domain triggers no or essentially no antibody mediated phagocytosis.

54. The polypeptide of any one of claims 1-53, wherein the Fc domain triggers no or essentially no antibody mediated cell cytotoxicity.

55. The polypeptide of any one of claims 1-54, wherein the Fc domain causes no or essentially no induction of activating FcγR.

56. A nucleic acid encoding any of the polypeptides of claim 1-55.

57. The nucleic acid of claim 56, wherein the nucleic acid is a DNA segment.

58. The nucleic acid of claim 56, wherein the nucleic acid is an expression vector.

59. A host cell comprising the nucleic acid of any one of claims 56-58.

60. The host cell of claim 59, wherein the host cell expresses the nucleic acid.

61. The host cell of any one of claims 59-60, wherein the host cell is a eukaryotic cell.

62. The host cell of any one of claims 59-60, wherein the host cell is a mammalian cell, an insect cell, or a yeast cell.

63. A method for preparing an aglycosylated polypeptide comprising:
a) obtaining a host cell in accordance with any one of claims 60-62;
b) incubating the host cell in culture under conditions to promote expression of the aglycosylated polypeptide; and
c) purifying the expressed polypeptide from the host cell.

64. The method of claim 63, wherein the host cell is a eukaryotic cell.

65. The method of claim 64, wherein the host cell is a mammalian cell, insect cell, or a yeast cell.

66. A pharmaceutical formulation comprising a polypeptide of any one of claims 1-55, or the nucleic acid of any one of claims 56-58 in a pharmaceutically acceptable carrier.

67. A method of binding a protein in a mammalian subject comprising administering to the subject an antibody, wherein the antibody is binds the protein and comprises an Fc domain according to any one of claims 1-55.

68. The method of claim 67, wherein the antibody is capable of specifically binding human FcγRIIb, and wherein the antibody has a reduced binding of one or more activating Fcγ receptors as compared to a human wild-type IgG Fc domain.

69. The method of claim 68, wherein the antibody is aglycosylated.

70. The method of claim 68, wherein the antibody is glycosylated.

71. The method of any one of claims 67-70, wherein the antibody results in no or essentially no antibody-mediated phagocytosis in the subject after the administering.

72. The method of any one of claims 67-71, wherein the mammalian subject is a human.

73. The method of any one of claims 67-72, wherein the antibody binds FcγRIIa_{R131} receptor in the subject with an affinity that is at least about 30-fold less than or about 40-fold less than a wild-type Fc.

74. The method of any one of claims 67-73, wherein the antibody does not selectively or detectably bind one or more activating human Fcγ receptor polypeptide in the subject.

75. The method of any one of claims 73-74, wherein the activating human Fcγ receptor polypeptide is FcγRI, FcγRIIa H131, FcγRIIIa F158, and/or FcγRIIIa V158.

76. The method of any one of claims 67-75, wherein the antibody does not specifically or detectably bind one or more activating human C1q.

77. The method of claim 67, wherein the antibody is an aglycosylated version of a therapeutic antibody.

78. A method of treating a subject having a disease comprising administering to the subject an effective amount of the formulation of claim 66.

79. The method of claim 71, wherein the method does not induce antibody-dependent cytotoxicity.

80. The method of claim 78, wherein the disease is a cancer, an infection, or an autoimmune disease.

81. The method of claim 78, wherein the subject is a human patient.
